(19) United States
(12) Patent Application Publication
(10) Pub. No.: US 2007/0180542 A1
(43) Pub. Date: Aug. 2, 2007

(54) Culture conditions and growth factors affecting fate determination, self-renewal and expansion of rat spermatogonial stem cells

(75) Inventors: Ralph L. Brinster, Gladwyne, PA (US); Hiroshi Kubota, Swarthmore, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Trustees of the University of Pennsylvania

(21) Appl. No.: 11/546,503

(22) Filed: Oct. 11, 2006

Related U.S. Application Data
(63) Continuation of application No. PCT/US05/12273, filed on Apr. 11, 2005.
(60) Provisional application No. 60/561,464, filed on Apr. 12, 2004.
(60) Provisional application No. 60/598,148, filed on Aug. 2, 2004.

(51) Int. Cl. A01K 67/027; A01N 1/02; G01N 33/567

(52) U.S. Cl. 800/14; 435/7.2; 435/2; 800/21; 800/17

(57) ABSTRACT

The present invention relates to methods of identifying and enriching spermatogonioal stem cells, and compositions thereof. Further, the invention relates to methods and compositions for the isolation, maintenance and proliferation of spermatogonial stem cells, as well as methods and compositions for the identification and use of factors influencing spermatogonial stem cell maintenance and proliferation.

### CROSS-REFERENCE TO RELATED APPLICATIONS

This application is a continuation of PCT International Application No. PCT/US2005/12273, filed Apr. 11, 2005, which in turn claims the benefit pursuant to 35 U.S.C. §119(e) of U.S. Provisional Application Nos. 60/561,464, filed Apr. 12, 2004 and 60/598,148, filed Aug. 2, 2004, all of which are hereby incorporated by reference in their entirety herein.

### STATEMENT REGARDING FEDERALLY SPONSORED RESEARCH OR DEVELOPMENT

This invention was supported in part by US Government funds (National Institutes of Health grant No. RO1 HD044445), and the US Government may therefore have certain rights in the invention.

### BACKGROUND OF THE INVENTION

Mammalian spermatogonial stem cells (SSCs) self-renew and produce daughter cells that commit to differentiate into spermatozoa throughout adult life of the male (Meistrich et al., Oxford Univ. Press; 266-295 (1993)). SSCs can be identified unequivocally by a functional assay using a transplantation technique in which donor testis cells are injected into the seminiferous tubules of infertile recipient males (Brinster et al., Proc. Natl. Acad. Sci. U.S.A., 91:11298-302 (1994), Brinster et al., Proc. Natl. Acad. Sci. U.S.A., 91:11303-7 (1994)). Under these conditions, only SSCs are able to generate colonies of complete spermatogenesis and restore long-term normal spermatogenesis. Although SSCs and the surrounding microenvironment have been studied during the past decade using the transplantation assay (Brinster et al., Science, 296:2174-6 (2002)), mechanisms underlying the process of self-renewal and differentiation of SSCs remain elusive. One approach to the problem is cultivation of SSCs under conditions that allow self-renewal and possibly inducible differentiation. For this purpose, it is essential to establish a culture system with defined, experimentally modifiable characteristics.

Serum-free culture systems (i.e., culture systems that do not contain serum) are an important approach to investigate the biological properties of mammalian cells in vitro (Barnes et al., Cell, 22:649-655 (1980), Ham et al., Methods Enzymol., 58:44-93.: 44-93 (1979)). Serum contains complex undefined materials, and batch variations occur depending on many uncontrollable factors, for example the physiological condition or sex of donors. In addition, substances in serum are toxic for certain cell types (Barnes et al., Cell, 22:649-655 (1980), Enat et al., Proc. Natl. Acad. Sci. U.S.A., 81:1411-5 (1984)). Once mammalian cells were shown to proliferate in serum-free hormonally defined medium without altering the cell type-specific characteristics (Hayashi et al., Nature, 259:132-134 (1976)), serum-free culture became a major resource to study cells in vitro and to identify novel growth factors or regulatory mechanisms for proliferation and differentiation. Using serum-free culture systems, it was determined that most cell types require specific growth factors and hormones to proliferate in vitro (Barnes et al., Cell, 22:649-655 (1980), Hayashi et al., Nature, 259:132-134 (1976)). In culture studies using SSCs, serum has been used at various concentrations, perhaps because embryonic stem (ES) cells have generally been maintained with high concentrations of serum. In early reports on the culture of SSCs, media contained 10% fetal bovine serum (FBS), and some SSCs survived for more than 3 months (Nagano et al., Tissue Cell, 30:389-97 (1998)). A similar concentration of serum was present in media testing the effect of growth factors and various feeder cell types (Nagano et al., Biol. Reprod., 68:2207-2214 (2003)). Recently, long-term survival and proliferation of SSCs was reported in a proprietary medium (Stem Pro-34 SFM; Invitrogen, Carlsbad, Calif.) with 1% FBS and mouse embryonic feeder cells (Kanatsu-Shinohara et al., Biol. Reprod., 69:612-616 (2003)). While this medium contains serum and is not defined, the long-term proliferation of SSCs in vitro is a significant development. A major challenge still remaining is to establish a defined serum-free culture condition that supports maintenance of the stem cell and allows definitive experiments to analyze the effect of individual medium modifications on proliferation.

Cell fate determination between self-renewal or differentiation of SSCs in the testis is precisely regulated to maintain normal spermatogenesis. Fate determination of stem cells is controlled to a large extent by the surrounding microenvironment, particularly the stem cell niche (Spradling et al., Nature, 414:98-104 (2001)). Little is known about the components of the stem cell niche. However, studies with hematopoietic stem cells suggest that feeder cells are an essential element to reconstitute stem cell niches in vitro (Moore et al., Blood, 89:337-4347 (1997)). Likewise, co-culture with mouse fibroblast cell line STO (“STO”) cell feeders improved in vitro maintenance of SSCs compared to no feeders, although the co-culture system maintained only 10 to 20% of stem cells for 7 days (Nagano et al., Tissue Cell, 30:389-97 (1998)), (Nagano et al., Biol. Reprod., 68:2207-2214 (2003)). This result suggests that STO cell feeders, which can support ES cells (Robertson Oxford, England: IRS Press, 71-112 (1987)), might reconstitute a stem cell niche for SSCs in vitro. However, since crude cryptorchid testis cell populations were used as a stem cell source in the study, it is not clear whether STO cell feeders alone provide the beneficial effects on SSCs survival or the combination of STO cells and testis cells was necessary. To avoid ambiguity associated with the diverse testis somatic cell population on SSC maintenance, it is important to use highly enriched SSCs for in vitro culture studies.

Because SSCs are rare in the testis, presumably 1 in 3000 to 4000 cells in adult mouse testis (Tegelenbosch et al., Mutat. Res., 290:193-200 (1993)), several approaches to enrich stem cells have been attempted. Experimental cryptorchid surgery resulted in approximately a 20 to 25-fold enrichment of SSCs (Shinohara et al., Dev. Biol., 220:401-11 (2000)). Cell suspensions from cryptorchid mouse testis contained about one SSCs in 200 cells. In addition, immunological separation using surface antigenic properties is a major approach for enrichment of SSCs (Kubota et al., PNAS., 100:6487-6492 (2003), Shinohara et al., Proc. Natl. Acad. Sci. U.S.A., 97:8346-51 (2000)), as has been shown in other stem cell systems (Kubota et al., Proc. Natl. Acad. Sci. U.S.A., 97:12132-7 (2000), Spangrude et al., Science, 241:58-62 (1988)). To obtain a pure or highly enriched stem cell population, it is critical to identify unique surface makers that are expressed on stem cells, because the antigenic profile of stem cells establishes the basis for selective separation. Particularly, identification of surface markers that are expressed uniquely on SSCs, but not on other somatic cells or differentiated spermatogenic cells facilitates enrichment of SSCs. It is also important to establish that expression of stem cell markers is conserved during development, indicating possible association with biological properties of the stem cells.

In a study in mice, Thy-1 was identified as a positive marker expressed uniquely on SSCs (Kubota et al., PNAS., 100:6487-6492 (2003)). Thy-i is a glycosyl phosphatidylinositol anchored surface antigen and is expressed on other stem cells including hematopoletic stem cells, mesenchymal stem cells, or ES cells (Spangrude et al., Science, 241:58-62 (1988), Henderson et al., Stem Cells, 20:329-337 (2002), Pittenger et al., Science, 284:143-147 (1999)). The study indicates that major histocompatibility complex class I (MHC-I)-Thy-1+c-kit cells isolated by flow cytometric sorting from experimental cryptorchid testis cells contained SSCs at a concentration of 1 in 15 cells and that the MHC-- Thy-1 c-kit cells contained almost all the SSCs in the testis (Kubota et al., PNAS., 100:6487-6492 (2003)). Since most of the MHC-I-Thy-1+cells in the testis were c-kit-, Thy-1 antigen is a key molecule to enrich SSCs. However, the expression of Thy-i on SSCs in neonate or pup testis has not been examined. Therefore, it is unclear as to whether SSCs express Thy-i constitutively throughout postnatal life. Although the concentration of SSCs appears to be lower in neonatal and pup testes than in cryptorchid (Shinohara et al., Proc. Natl. Acad. Sci. U.S.A., 98:6186-91 (2001)), it has not been determined whether stem cell activity of SSCs enriched by a common characteristic from neonate, pup, and adult testes are identical.

Because there is currently a deficit in the understanding of the mechanisms underlying the regulation of spermatogenesis, there is a need for a better understanding of the factors controlling the cell fate determination of SSCs. Accordingly, there is a need to identify in vitro conditions and parameters that will enable the identification of factors that regulate the growth and maintenance of SSCs. Further, in order to study SSCs in this manner, and in order to generate SSCs-based therapeutic treatments, there is a need to identify methods of enriching a population of SSCs. The present invention meets these needs.

### SUMMARY OF THE INVENTION

In an embodiment, the present invention features a method of enriching spermatogonial stem cells (SSCs) from a population of testis-derived cells containing at least one SSC. The method includes providing an antibody specific for the SSC cell-surface marker Thy-1, contacting a population of testis-derived cells with the antibody under conditions suitable for formation of an antibody-SSC complex, and substantially separating the antibody-SSC complex from the population of testis-derived cells In another embodiment, the invention features a method of enriching spermatogonial stem cells (SSCs) from a population of testis-derived cells containing at least one SSC, wherein the method includes providing an antibody specific for the SSC cell surface marker a6-integrin, contacting a population of testis-derived cells with the antibody under conditions suitable for formation of an antibody-SSC complex, and substantially separating the antibody-SSC complex from the population of testis-derived cells.

In an embodiment, the invention also features a method of enriching spermatogonial stem cells (SSCs) from a population of testis-derived cells containing at least one SSC, wherein the method includes the steps of providing a first antibody specific for the SSC cell surface marker Thy-1, providing a second antibody specific for an SSC cell surface marker other than Thy-1, contacting a population of testis-derived cells with the first antibody under conditions suitable for formation of an antibody-SSC complex, substantially separating the first antibody-SSC complex from the population of testis-derived cells, thereby creating a first antibody-SSC complex population of cells, contacting the first antibody-SSC complex population of cells with the second antibody under conditions suitable for formation of a second antibody-SSC complex, substantially separating the second antibody-SSC complex from the population of testis-derived cells.

In one aspect of the invention, an SSC is a human SSC. In another aspect, an SSC is derived from an organism selected from the group consisting of a mouse, a rat, a monkey, a baboon, a cow, a pig and a dog.

In another aspect of the invention, cells are derived from a source selected from the group consisting of mouse wild type adult testis, mouse pup testis, mouse neonate testis, and mouse cryptorchid adult testis.

In one embodiment of the invention, an antibody is selected from the group consisting of an isolated antibody, a biological sample comprising an antibody, an antibody bound to a physical support and a cell-bound antibody. In another aspect of the invention, an antibody is selected from the group consisting of a polyclonal antibody, a monoclonal antibody, a humanized antibody, a synthetic antibody, and combinations thereof, or biologically active fragments, functional equivalents, derivatives, and allelic or species variants thereof. In another aspect of the invention, a biologically active antibody fragment is selected from the group consisting of a Fab fragment, a F(ab′)₂ fragment, and a Fv fragment.

In an aspect of the invention, a physical support is selected from the group consisting of a microbead, a magnetic bead, a panning surface, a dense particle for density centrifugation, an adsorption column and an adsorption membrane.

In one embodiment of the invention, an antibody-SSC complex is substantially separated from said population of testis-derived cells by a method selected from the group consisting of fluorescence activated cell sorting (FACS) and magnetic activated cell sorting (MACS).

In an embodiment, the invention also features a method of detecting an SSC in a population of testis-derived cells, wherein the method includes providing an antibody specific for Thy-1, contacting the population of testis-derived cells with the antibody under conditions suitable for formation of an antibody-SSC complex, and detecting the antibody-SSC complex. In another embodiment, the invention features a method of detecting an SSC in a population of testis-derived cells, wherein the method includes providing an antibody specific for at least one cell surface marker selected from the group consisting of Thy-1, epithelial glycoprotein-2 (EpCAM), neural cell adhesion molecule (NCAM), glial cell-derived neurotrophic factor family receptor alpha-I (GFRAI) and cell adhesion marker CD24 (CD24), contacting the population of testis-derived cells with the antibody under conditions suitable for formation of an antibody-SSC complex, and detecting the antibody-SSC complex.

In an embodiment, the invention features a serum-free culture system for support of SSC maintenance, the system comprising enriched SSCs, serum-free defined culture medium, and mitotically-inactivated fibroblast feeder cells. In another embodiment, the invention features a serum-free culture system for support of SSC proliferation comprising at least one SSC, serum-free defined culture medium, and mitotically-inactivated mouse fibroblast cell line STO (“STO”) feeder cells.

In one aspect of the invention, a culture system further comprises at least one growth factor selected from the group consisting of SCF, GDNF, GFRXI, LIF, bFGF, EGF and IGF-I. In another aspect, a culture medium comprises at least one medium selected from the group consisting of minimal essential medium-alpha (MEMa), Ham's FIO culture medium, RPMI bicarbonate-buffered medium, and Dulbecco's MEM: Ham's Nutrient Mixture F-12 (DMEM/F12).

In an embodment, the invention features a composition comprising a population of enriched SSCs, wherein the enriched SSCs express a Thy-i marker. In another embodiment, the invention features a composition comprising a population of Thy-l-enriched SSCs. In one aspect, a population of Thy-1-enriched SSCs is substantially homogeneous for SSCs expressing a Thy-i marker. In another aspect, a population of enriched SSCs is substantially homogeneous for SSCs expressing a Thy-1 marker.

In an embodiment, the invention features a method of generating at least one mammalian progeny, comprising administering a population of Thy-1-enriched SSCs to a testis of a male recipient mammal, allowing the enriched SSCs to generate a colony of spermatogenesis in the recipient mammal, and mating the recipient mammal with a female mammal of the same species as the recipient mammal. In one aspect, a population of enriched SSCs is administered to the lumen of a seminiferous tubule of the recipient mammal. In another aspect, the recipient mammal is infertile.

In an embodiment of the invention, a recipient mammal is selected from the group consisting of a rodent, a primate, a dog, a cow, a pig and a human. In another embodiment, a rodent is selected from the group consisting of a mouse and a rat. In yet another aspect, the primate is a baboon.

In one embodiment, the invention features a method of generating at least one progeny mammal, comprising administering a population of enriched SSCs to a testis of a male recipient mammal, allowing the enriched SSCs to generate a colony of spermatogenic cells in the recipient mammal, and mating the recipient mammal with a female mammal of the same species as the recipient mammal.

In another embodiment of the invention, a method of determining the effect of a growth factor on an SSC includes providing a serum-free SSC culture system comprising a first population of enriched SSCs, serum-free defined culture medium, and a population of mitotically inactivated STO feeder cells, contacting the culture system with at least one growth factor, assessing the activity of the first population of enriched SSCs, and comparing the activity of the first population of enriched SSCs with a second population of enriched SSCs, wherein the second population of enriched SSCs is cultured in a growth factor-free culture system that is otherwise identical to the culture system comprising the first population of enriched SSCs, wherein a higher level of SSC activity in the population of first enriched SSCs is an indication that the growth factor enhances the activity of an SSC.

In one embodiment, the invention features a method of determining the effect of a growth factor on an SSC, comprising providing a serum-free SSC culture system comprising a first population of enriched SSCs, serum-free defined culture medium, and a population of mitotically inactivated STO feeder cells, contacting the culture system with at least one growth factor, assessing the activity of the first population of enriched SSCs, and comparing the activity of the first population of enriched SSCs with a second population of enriched SSCs, wherein the second population of SSCs is cultured in a growth factor-free culture system that is otherwise identical to the culture system comprising the first population of enriched SSCs. The method further provides that a lower level of SSC activity in the population of first enriched SSCs is an indication that the growth factor inhibits the activity of an SSC.

In one aspect of the invention, a growth factor is selected from the group consisting of bFGF, IGF 1, GDNF and GFRa1. In another aspect, a growth factor is selected from the group consisting of LIF, bFGF, EGF and IGF-I.

The invention also features a method of maintaining at least one SSC in a serum-free culture system, wherein the method includes providing a culture system comprising serum-free defined culture medium and mitotically-inactivated STO feeder cells; adding at least one enriched SSC to the culture system.

In another embodiment, the invention features a method of maintaining at least one SSC in a serum-free culture system, including providing a culture system comprising serum-free defined culture medium and mitotically-inactivated STO feeder cells, adding at least one enriched SSC to the culture system, and essentially eliminating inhibitory testis somatic cells and germ cells from the culture system.

The invention also features a method of proliferating at least one SSC in a serum-free culture system, comprising providing a culture system comprising serum-free defined culture medium and mitotically-inactivated STO feeder cells, and adding at least one enriched SSC to said culture system. In another embodiment, the invention features a method of proliferating at least one SSC in a serum-free culture system, the method comprising providing a culture system comprising serum-free defined culture medium and mitotically-inactivated STO feeder cells, adding at least one enriched SSC to the culture system, and essentially eliminating inhibitory testis somatic cells and germ cells from the culture system.

In an embodiment, the present invention also features a method of proliferating at least one SSC in a serum-free culture system, comprising providing a culture system comprising serum-free defined culture medium and mitotically-inactivated STO feeder cells, adding at least one enriched SSC to said culture system, and contacting the enriched SSC with GDNF.

In another embodiment, the invention also features a method of proliferating at least one SSC in a serum-free culture system, comprising providing a culture system comprising serum-free defined culture medium and mitotically-inactivated STO feeder cells, adding at least one SSC to the culture system, and stimulating at least one GDNF cell-signaling pathway in the SSC. In one aspect, the SSC is an enriched SSC.

The present invention also features a method of proliferating at least one SSC in a serum-free culture system, comprising providing a culture system comprising serum-free defined culture medium and mitotically-inactivated STO feeder cells, adding at least one SSC to the culture system, and stimulating at least one GDNF cell-signaling pathway in the SSC, wherein the stimulation of the GDNF cell-signaling pathway is effected by using at least one of the factors selected from the group consisting of GDNF, GFRal and bFGF. In one aspect, the SSC is an enriched SSC.

In an embodiment, the present invention features a method of proliferating at least one SSC in a culture system, wherein the method includes providing a culture system comprising a culture medium and mitotically-inactivated STO feeder cells, and adding at least one enriched SSC to the culture system. In another embodiment, the invention features a method of proliferating at least one SSC in a culture system, wherein the method includes providing a culture system comprising a culture medium and mitotically-inactivated STO feeder cells, adding at least one enriched SSC to said culture system, and essentially eliminating inhibitory testis somatic cells and germ cells from the culture system.

The invention also features a method of proliferating at least one SSC in a culture system, comprising providing a culture system comprising a culture medium and mitotically-inactivated STO feeder cells, adding at least one enriched SSC to the culture system, and contacting the enriched SSC with GDNF. In another aspect, the invention features a method of proliferating at least one SSC in a culture system, comprising providing a culture system comprising a culture medium and mitotically-inactivated STO feeder cells, adding at least one enriched SSC to the culture system, and stimulating at least one GDNF cell-signaling pathway in the enriched SSC.

In an embodiment, the invention also features a method of proliferating at least one SSC in a culture system, wherein the method includes providing a culture system comprising a culture medium and mitotically-inactivated STO feeder cells, adding at least one enriched SSC to the culture system, and stimulating at least one GDNF cell-signaling pathway in said enriched SSC, wherein the stimulation of the GDNF cell-signaling pathway is effected by using at least one of the factors selected from the group consisting of GDNF, GFRal and bFGF.

In another embodiment, the invention features a kit for maintaining at least one SSC in a serum-free culture system. The kit includes a culture system comprising serum-free defined culture medium and mitotically-inactivated STO feeder cells, an applicator, and instructional material, wherein the instructional material comprises instructions for the use of the kit to maintain at least one SSC in the serum-free culture system. In yet another embodiment, A kit for proliferating at least one SSC in a serum-free culture system, comprising a culture system comprising serum-free defined culture medium and mitotically-inactivated STO feeder cells, an applicator, and instructional material, wherein the instructional material comprises instructions for the use of the kit to proliferate at least one SSC in said serum-free culture system.

In yet another embodiment, the invention provides a kit for administering a population of enriched SSC to a mammal. The kit includes a culture system comprising serum-free defined culture medium and mitotically-inactivated STO feeder cells, an applicator, and instructional material, wherein the instructional material includes instructions for the use of the kit to proliferate at least one SSC in a serum-free culture system, and instructions for the applicator-based administration of enriched SSC to a mammal.

In an embodiment, the invention features a progeny animal produced according to a method of the invention. In another embodiment, the invention features a progeny animal made according to a method of the invention, wherein the enriched SSCs used to make the progeny animal contain at least one genetic mutation. In one aspect, a genetic mutation is created using recombinant techniques.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1D are a series of images depicting flow cytometric analysis of cryptorchid adult testis cells and stem cell activity of subpopulations of the testis isolated by FACS.

FIG. 1A is an image of a staining profile of 02M versus Alexa Fluor 488-SAv for cryptorchid adult testis cells. P2M expression was detected with Alexa Fluor 647-conjugated secondary antibody.

FIG. 1B is an image of a staining profile of 02M versus Thy-i (B) for cryptorchid adult testis cells. Thy-1 expression was detected with Alexa Fluor 488-SAv.

FIG. 1C is a graph depicting colonization of recipient testes by transplanted ROSA donor testis cells. Cells from three fractions (G1, G2, and G3) in the experiment described in FIG. 1 B were sorted and transplanted into infertile mouse testes to determine stem cell activity. The number of spermatogenic colonies generated by 10⁵ cells transplanted to recipient testis was: G1, 282.6 30.4, n=9; G2, 6.5±2.4, n=10; G3, 0.8±0.8, n=12; unsorted cells, 18.6+3.9, n=10 (mean+SEM). Three gates were created based on the expression profile of 02M and Thy-1 in the experiments depicted in FIGS. 1A and 1B. G1, G2, and G3 represent P2M Thyl+, 02M Thy-1, and 02M+cells, respectively. Gated cell distribution of the results of the experiments described in FIG. 1A is: G1, 0.5%; G2, 12.5%; G3, 85.5%; and in FIG. 1B is: G1, 6.4%; G2, 4.3%; G3, 87.5%.

FIG. 1 D is a series of images depicting the macroscopic appearance of recipient testes 2 months after transplantation with sorted testis cells from GI (Left panel), G2 (Center panel), and G3 (Right panel). Each (blue) contrast-stained stretch of cells in the recipient testis represents a donor-derived spermatogenic colony. Stain =X-gal. Bar=2 mm.

FIGS. 2A-2C are a series of images depicting the flow cytometric analysis of side scatterlow P2M wild type adult testis cells and stem cell activity of subpopulations of the testis isolated by FACS. Wild type adult testis cells from bottom fraction after Percoll separation were stained with anti- ,P2M, anti-a6-integrin, 5 and anti-Thy-i antibodies.

FIG. 2A is an image depicting the staining profile of a6-integrin versus isotype control for side scattelow P2M wild type adult testis cells.

FIG. 2B is an image depicting the staining profile of a6-integrin versus Thy-1 for side scatterloW P2M wild type adult testis cells. 10 FIG. 2C is a graph depicting the degree of colonization from sorted cells of the experiment described in FIG. 2B, with the degree of colonization represented by the number of individual blue spermatogenic colonies. Cells from B were sorted into three groups (G1, G2, and G3) based on the expression profile of a6-integrin and Thy-l. G1, G2, and G3 represent a6-integrin+Thy-1+, a6-integrin+Thy-1i5 , and a6-integrin cells, respectively. Cell were then transplanted into infertile mouse testes to determine stem cell activity. The number of spermatogenic colonies generated by 105 cells transplanted to recipient testis was: G1, 161.6+50.5, n 10;

G2, 10.2±2.7, n=10; G3, 00, n=10; unsorted cells of Percoll-bottom fraction, 17.9±3.4, n =12 (mean±SEM). The gated cell distribution of cells from the 20 experiment described in FIG. 2A was: G1, 0.4%; 62, 24.9%; G3, 66.6%; and from FIG. 2B was: G1, 2.6%; G2, 18.6%; G3, 70.6%.

FIGS. 3A-3C are a series of images depicting the flow cytometric analysis of pup testis cells and stem cell activity of subpopulations of the testis isolated. by FACS. 25 FIG. 3A is an image depicting the staining profile of av-integrin versus Alexa Fluor 488-SAv for pup testis cells.

FIG. 3B is an image depicting the staining profile of av-integrin versus Thy-1 (B) for pup testis cells. Alexa Fluor 488-SAv was used to detect biotin-Thy-1 antibody. 30 FIG. 3C is a graph depicting the degree of colonization from sorted cells in the experiment set forth in FIG. 3B, and is represented by the number of individual blue spermatogenic colonies. Three gates were created based on the expression profile of av-integrin and Thy-1. GI, G2, and G3 represent av-integrin Thy-i+, av-integrin Thy-1, and av-integrin+cells, respectively. Gated cell distribution of the experiment set forth in FIG. 3A was: G1, 0.3%; 02, 10.0%; G3, 86.2%; and in FIG. 3B was: G1, 5.6%; G2, 4.8%; G3, 87.2%. Cells from the experiment set forth in FIG. 3B were sorted into three fractions (GI, G2, and G3) and transplanted into infertile mouse testes to determine stem cell activity. The number of spermatogenic colonies generated by 1 cells transplanted to recipient testis was: G1, 124.2 17.2, n=11; G2, 2.4±1.2, n=12; G3, 1.5±0.9, n =12; unsorted cells, 12.9±2.3, n =10 (mean±SEM).

FIGS. 4A-4C are a series of images depicting the flow cytometric analysis of neonate testis cells and stem cell activity of subpopulations of the testis isolated by FACS.

FIG. 4A is an image of a staining profile of av-integrin versus Alexa Fluor 488-SAv (A) for pup testis cells.

FIG. 4B is an image of a staining profile of av-integrin versus Thy-l (B) for pup testis cells.

FIG. 4C is a graph depicting the degree of colonization from transplanted donor pup cells from the experiment set forth in FIG. 4B, and is represented by the number of individual blue spermatogenic colonies. Cells from the experiment set forth in FIG. 4B were sorted into three fractions (G1, G2, and G3) and transplanted into infertile mouse testes to determine stem cell activity. The number of spermatogenic colonies generated by 10⁵ cells transplanted to recipient testis was: G1, 17.3+5.7, n =12; G2, 0.0±0.0, n=12; G3, 0.0 +0.0, n=12; unsorted cells, 0.8±0.3, n =12 (mean±SEM). Three gates were created based on the expression profile of av-integrin and Thy-1. G1, G2, and G3 represent av-integrin Thy-1+, av-integrinThy-l+, and av-integrin+cells, respectively. Gated cell distribution of data from the experiment set forth in FIG. 4A was: G1, 0.0%; G2, 9.0%; G3, 88.6%; and from the experiment set forth in FIG. 4B was: G1, 1.4%; G2, 7.6%; G3, 88.6%.

FIG. 5 is a graph depicting the enrichment of spermatogonial stem cells by Thy-1 antibody-conjugated microbeads. The degree of colonization from Thy-1 microbead-selected or freshly isolated ROSA donor testis cells is represented by the number of individual blue spermatogenic colonies per 105 cells transplanted. The donor testis cells were isolated from cryptorchid adult, wild type adult, pup, and neonate testes. The number of spermatogenic colonies generated by 105 cells transplanted to recipient testis was: cryptorchi adult, 191.9±21.7, n =18 (U), 31.2 +5.9, n =16(o); wild type adult, 48.1±11.8, n=12(u), 1.6±0.3, n=1 (o); pup, 69.6±9.5, n=12(n), 14.4+4.4, n=9 (o); neonate, 21.8±4.6, n =18 (U), 1.7±0.3, n =17 (o), (mean±SEM).

FIG. 6 is a graph depicting the effect of fetal bovine serum, basal medium type, and feeder cells on maintenance and proliferation of spermatogonial stem cells in culture. 02M Thy-1+cells from ROSA cryptorchid testis cells isolated by FACS were cultured for 8-10 days in the conditions indicated. After in vitro culture, donor cells were harvested and transplanted into recipient testes. The degree of colonization of the recipient testis is represented by the number of spermatogenic colonies per 105 donor P 2M Thy-+cells originally seeded in culture. P2M Thy-1+cells prior to culture generated 282.6±30.4 (n =9) colonies per 10⁵ cells in recipient testes (FIG. 1 C). The number of colonies from donor cells cultured with STO feeders in MEMa medium supplemented with 10% FBS (500.0+76.8, n =9) increased comparing to the 282.6 value. Data are presented as mean±SEM, and nine to twelve recipient testes were analyzed per group.

FIG. 7 is a graph depicting the in vitro maintenance and proliferation of spermatogonial stem cells enriched by MACS using Thy-1 antibody-conjugated magnetic microbeads. Enriched spermatogonial stem cells (MACS Thy-1+cells) were cocultured with STO feeders in FBS (iO%)-supplemented or serum-free condition using MEMa-based medium. Freshly isolated MACS Thy-1+cells, one-week cultured cells, and two-week cultured cells were transplanted into recipient testes. The number of donor derived spermatogenic colony per 105 MACS Thy-1+cells (Fresh) or per 10⁵ MACS Thy-1+cells originally seeded in culture (1 week and 2 weeks) is presented.

FIG. 8 is a series of graphs depicting the effect of growth factors on maintenance and proliferation of spermatogonial stem cells in a serum-free defined medium. MACS Thy-1+cells were cultured with STO feeders in a MEMo-based serum-free medium for 7 days with the growth factor indicated at 2 to 3 concentrations. Cultured cells were harvested after one week and transplanted into recipient testes. The degree of colonization of the recipient testis is represented by relative colonization activity, the number of colonies per 105 donor cells originally placed in culture relative to that obtained with the control culture at the concentration of 0 ng/ml or 0 unit/ml of each growth factor. Data are presented as means±SEM, and five to twelve recipient testes were analyzed per group.

FIGS. 9A-9E are a series of images illustrating the expansion of DBAxROSA SSCs in serum-free medium supplemented with GDNF.

FIG. 9A is an image illustrating that DBAxROSA pup testis cells formed clumps with tight intercellular contacts in culture. Bar =100 μm.

FIG. 9B is an image illustrating that cultured cells in clumps express GCNA1, a marker for germ cells. Bar =100 μm.

FIG. 9C is an image illustrating that expression of P-gal was detected only in germ cell clumps, P-gal expressing cells stain blue with X-gal. Bar =100 μm.

FIG. 9D is an image illustrating the macroscopic appearance of recipient testis 2 months after or transplantation with DBAxROSA MACS Thy-i cells cultured for 1 0-weeks in the presence of GDNF. Each blue-stained area indicates donor-derived spermatogenesis. Stain =X-gal. Bar =2 mm.

FIG. 9E is a graph illustrating freshly isolated MACS Thy-1 cells and cultured cells were transplanted into recipient testes. The number of donor-derived spermatogenic colonies per 105 MACS Thy-1 cells originally seeded in culture is shown on the Y-axis. The transplantation assay demonstrated expansion of DBAxROSA SSCs in culture with GDNF. DBAxROSA SSCs cultured without GDNF and C57xROSA SSCs cultured with or without GDNF were not maintained. Data are presented as means±SEM, and 6 recipient testes were analyzed per time point.

FIGS. 10A-10E are a series of images depicting expansion of SSCs in serum-free medium supplemented with GDNF, soluble GFRal and bFGF.

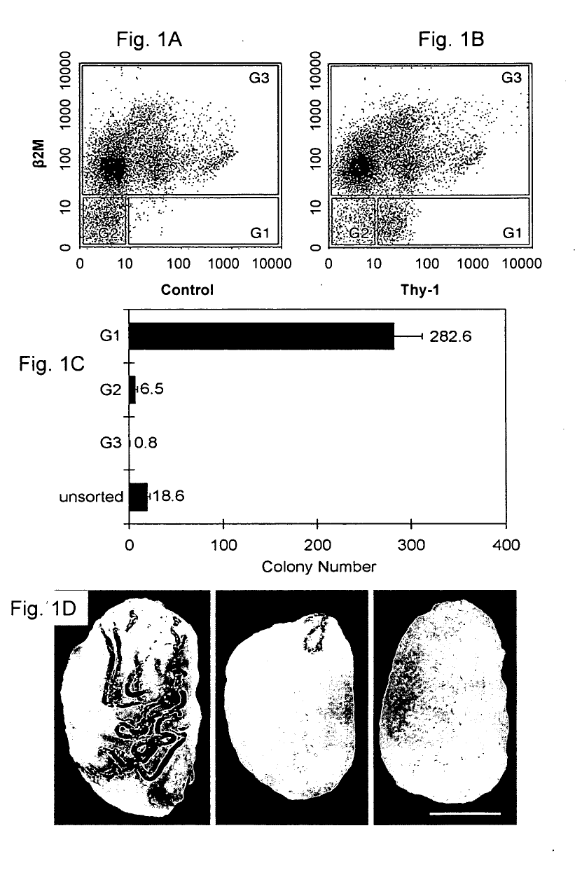

FIG. 1OA is a graph depicting MACS Thy-i cells from C57xROSA cultured in the conditions indicated in the figure legend. Fresh MACS Thy-1 cells and cultured cells were transplanted into recipient testes. The number of donor-derived spermatogenic colonies per i05 MACS Thy-1 cells originally seeded in culture is shown. The transplantation assay demonstrated a synergistic effect of soluble GFRal and bFGF on expansion of C57xROSA SSCs cultured with GDNF. Data are presented as means±SEM, and 6 recipient testes were analyzed per time point.

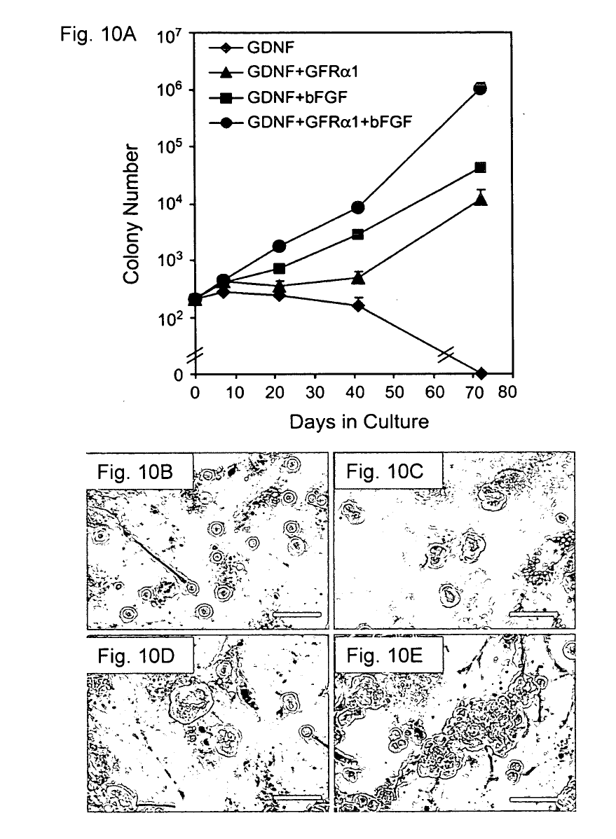

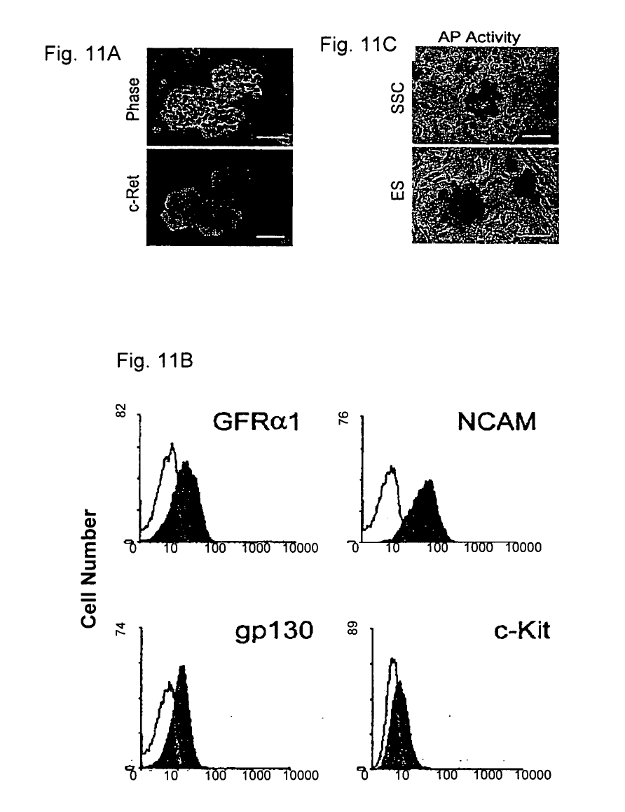

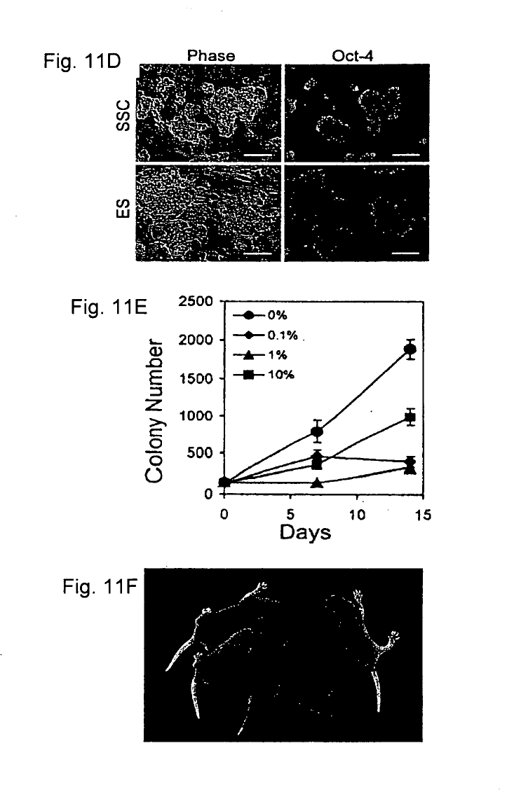

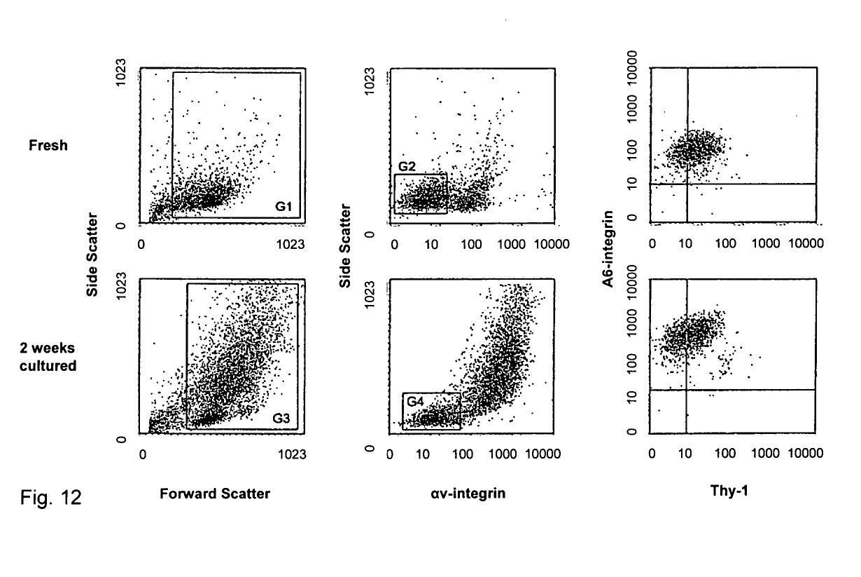

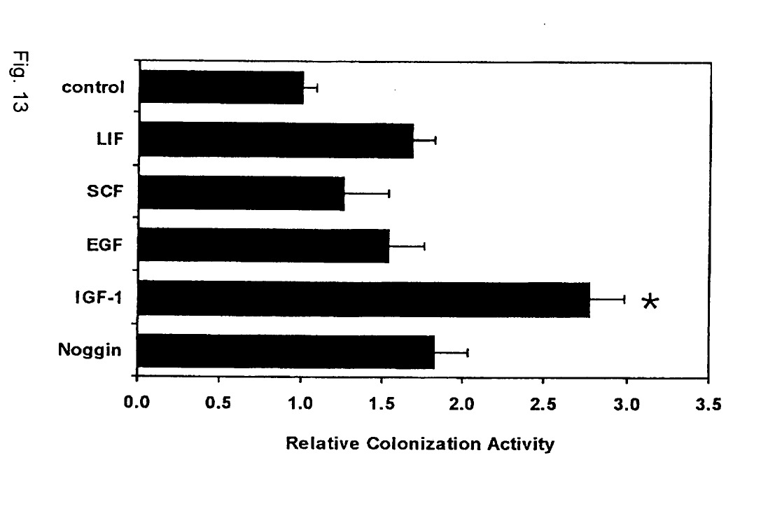

FIG. 10B is an image illustrating the development and growth of germ cell clumps from 129/SvCP MACS Thy-1 pup testis cells on STO feeders after 5 hours in culture. Bar =50 μm.

FIG. 10C is an image illustrating initiation of cell clump formation at 2 days. Bar =50 μm.

FIG. 10D is an image illustrating growth of germ cell clumps at 5 days. Bar=50 μm.

FIG. 10E is an image illustrating continuous expansion of germ cell clumps at 5 months. Bar =50 μm.

FIGS. 11A-11F are a series of images depicting the phenotypic and biological characteristics of cultured SSCs.

FIG. 11A is a pair of images illustrating the immunohistochemistry of c-Ret receptor tyrosine kinase. All cells in germ cell clumps express the c-Ret receptor. Bar =50 μm.

FIG. 11B is a series of plots depicting FACS analyses for GFRaL, NCAM, gp 130 and c-Kit expression on cultured germ cells. Closed histogram represents stained cells with the antibodies indicated. Open histogram indicates isotype control antibody-stained cells. Cultured SSCs expressed GFRaI, NCAM, and gp 130. Only very weak expression of c-Kit was observed.

FIG. 11C is a pair of images illustrating AP activity on SSCs and ES cells. Cultured germ cell clumps have lower AP activity than ES cells. Bar=100 μm.

FIG. 11D is a series of images depicting immunohistochemistry of Oct-4 on SSCs and ES cells. Germ cell clumps and ES cells express a high level of Oct-4. Bar =100 μm.

FIG. 11E is a graph depicting the effect of FBS on proliferation of SSCs. SSCs were exposed to PBS at the concentration indicated for 2 weeks. Cells were transplanted after 7 and 14 days of culture. At each time point, the number of colonies formed per 105 cells placed in culture is shown. Proliferation of SSCs was decreased in all concentrations of FBS compared to serum-free medium. All values are means±SEM, and 5-6 recipient testes were analyzed per time point.

FIG. l IF is an image depicting the restoration of fertility in infertile recipients by transplantation of cultured SSCs. Progeny from W mice transplanted with C57GFPxROSA-derived germ cell clumps. Because the transplanted SSCs are haploid for the GFP transgene, 50% of progeny should express GFP.

FIG. 12 is a series of images depicting flow cytometric analyses of fresh and cultured MACS Thy-1 cells from C57xROSA pup testes. Fresh MACS Thy-1 cells were stained with antibodies against av-integrin, a6:integrin and Thy-1 and analyzed by FACS (Top). Live cell population (G1) is analyzed for xv-integrin-dim expression (Top middle). About 70% of GI cells were av-integrindim. The xv-integrin-m cells (G2) were analyzed for (x6-integrin and Thy-1 expression (Top Right). Cells in G2 were a6-integrin+Thy-l+The fresh MACS Thy-i cells were cultured on STO feeders with GDNF, soluble GFRxl, and bFGF. After 2 weeks, the surface phenotype of the cultured cells was analyzed (Bottom). The live cell population (G3) contains two distinct populations. One cell population, side scatte cv-integrin+, represents STO feeder cells. The second cell population is side scatter lav-integrin-/di, (G4), which is germ cell clump-forming cells. Cells in G4 express a6-integrin and Thy-1 (Bottom Right), an expression pattern similar to av-integrin-ldim cells of fresh MACS Thy-1 cells (Top Right). The surface phenotype did not change during 6 months' culture.

FIG. 13 is a graph depicting the effect of soluble factors on proliferating SSCs in vitro. Soluble factors indicated were added individually in the culture of C57xROSA SSCs that were maintained with GDNF, soluble GFRal, and bFGP for several weeks. Control culture contained GDNP, soluble GFRal, and bFGP with no additional factors. After 6 weeks of culture with additional factors, cultured SSCs were harvested and transplanted into recipient testes to evaluate stem cell activity. The data are represented by relative colonization activity, the number of colonies per 105 donor cells originally placed in culture relative to that obtained with the control culture (means +SEM, n =10-12). A significant effect (asterisk) was observed in culture with IGF-l (2.77±0.68-fold increase, Bonferroni adjusted p-value <0.0005).

### DETAILED DESCRIPTION OF THE INVENTION

The present invention features methods and compositions for stem cell maintenance whereby a growth factor enables maintenance of initial SSC activity during the maintenance period. The invention further features methods and compositions for stem cell proliferation, whereby a growth factor enables proliferation of SSCs during the proliferation period. The methods and compositions described herein also provide a reproducible and powerful assay system to identify the effect of various environmental factors on SSC survival and replication in vitro.

The study of SSCs requires that SSCs can be reliably and repeatably identified, isolated and purified. One method of cell enrichment known in the art is the use of a cell surface marker that is unique to a single type of cell within a population of cells in order to identify a particular type of cell. The challenge in this type of cellular identification is identifying and defining such a unique marker. For SSCs, Thy-1 is such a marker. This is because it has been shown herein for the first time in the present invention that Thy-i is expressed as a surface marker on SSCs found in neonate, pup, and adult testis in mice. Thy-1 can therefore now be used to identify, isolate, purify and enrich SSCs.

SSC fate determination between self-renewal or differentiation of SSCs in the testis is precisely regulated to maintain normal spermatogenesis. Fate determination of stem cells is controlled to a large extent by the surrounding microenvironment, particularly the stem cell niche, but until the present invention, little was known about the components of the stem cell niche. It has been shown for the first time by way of the present invention that culture conditions including serum-free defined medium and STO feeder cells can be used to investigate and identify the factors contributing to the maintenance and proliferation of stem cells. Using the culture conditions and methods of the invention, an enriched Thy-l+SSC population can be maintained without significant loss of the stem cell activity during the culture period. This finding, set forth herein for the first time, represents a significant improvement over the 10-20% of stem cells maintained under previous serum-supplemented conditions using less-purified testis cell populations. Definitions Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed. according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The nomenclature used herein and the laboratory procedures used in analytical chemistry and organic syntheses described below are those well known and commonly employed in the art. Standard techniques or modifications thereof, are used for chemical syntheses and chemical analyses.

The articles “a” and “an” are used herein to refer to one or to more than one (i.e., to at least one) of the grammatical object of the article. By way of example, “an element” means one element or more than one element.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

As used herein, to “alleviate” a disease, disorder or condition means reducing the severity of one or more symptoms of the disease, disorder or condition.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.” Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.” A “portion” of a polynucleotide means at least at least about twenty sequential nucleotide residues of the polynucleotide. It is understood that a portion of a polynucleotide may include every nucleotide residue of the polynucleotide.

As used herein, the terms “gene” and “recombinant gene” refer to nucleic acid molecules comprising an open reading frame encoding a polypeptide. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, riucleic acid molecules encoding proteins from other species (homologs), which have a nucleotide sequence which differs from that of the human proteins described herein are within the scope of the invention. Nucleic acid molecules corresponding to natural allelic variants and homologs of a cDNA of the invention can be isolated based on their identity to human nucleic acid molecules using the human cDNAs, or a portion thereof, as a hybridization probe according to standard hybridization techniques under stringent hybridization conditions.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence. “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus. “Mutants,” “derivatives,” and “variants” of a polypeptide (or of the DNA encoding the same) are polypeptides which may be altered in one or more amino acids (or in one or more base pairs) such that the peptide (or nucleic acid) is not identical to the sequences recited herein, but has the same property as the wild type polypeptide.

A “variant” or “allelic or species variant” of a protein or nucleic acid is meant to refer to a molecule substantially similar in structure and biological activity to either the protein or nucleic acid. Thus, provided that two molecules possess a common activity and may substitute for each other, they are considered variants as that term is used herein even if the composition or secondary, tertiary, or quaternary structure of one of the molecules is not identical to that found in the other, or if the amino acid or nucleotide sequence is not identical.

A “recombinant polypeptide” is one which is produced upon expression of a recombinant polynucleotide.

By the term “specifically binds,” as used herein, is meant a compound, e.g., a protein, a nucleic acid, an antibody, and the like, which recognizes and binds a specific molecule, but does not substantially recognize or bind other molecules in a sample.

As used herein, to “treat” means reducing the frequency with which symptoms of a disease, disorder, or adverse condition, and the like, are experienced by a patient.

As the term is used herein, “modulation” of a biological process refers to the alteration of the normal course of the biological process. For example, modulation of the activity of a spermatogonial stem cell may be an increase in the activity of the cell. Alternatively, modulation of the activity of a spermatogonial stem cell may be a decrease in the activity of the cell. “Enriching,” as the term is used herein, refers to the process by which the concentration, number, or activity of something is increased from a prior state. For example, a population of 100 spermatogonial stem cells is considered to be “enriched” in spermatogonial stem cells if the population previously contained only 50 spermatogonial stem cells. Similarly, a population of 100 spermatogonial stem cells is also considered to be “enriched” in spermatogonial stem cells if the population previously contained 99 spermatogonial stem cells. Likewise, a population of 100 spermatogonial stem cells is also considered to be “enriched” in spermatogonial stem cells even if the population previously contained zero spermatogonial stem cells.

As the term is used herein, “population” refers to two or more cells.

As the term is used herein, “substantially separated from” or “substantially separating” refers to the characteristic of a population of first substances being removed from the proximity of a population of second substances, wherein the population of first substances is not necessarily devoid of the second substance, and the population of second substances is not necessarily devoid of the first substance. However, a population of first substances that is “substantially separated from” a population of second substances has a measurably lower content of second substances as compared to the non-separated mixture of first and second substances.

In one aspect, a first substance is substantially separated from a second substance if the ratio of the concentration of the first substance to the concentration of the second substance is greater than about 1. In another aspect, a first substance is substantially separated from a second substance if the ratio of the concentration of the first substance to the concentration of the second substance is greater than about 2. In yet another aspect, a first substance is substantially separated from a second substance if the ratio of the concentration of the first substance to the concentration of the second substance is greater than about 5. In another aspect, a first substance is substantially separated from a second substance if the ratio of the concentration of the first substance to the concentration of the second substance is greater than about 10. In still another aspect, a first substance is substantially separated from a second substance if the ratio of the concentration of the first substance to the concentration of the second substance is greater than about 50. In another aspect, a first substance is substantially separated from a second substance if the ratio of the concentration of the first substance to the concentration of the second substance is greater than about 100. In still another aspect, a first substance is substantially separated from a second substance if there is no detectable level of the second substance in the composition containing the first substance. “Substantially homogeneous,” as the term is used herein, refers to a population of a substance that is comprised primarily of that substance, and one in which impurities have been minimized. “Maintenance” of a cell or a population of cells refers to the condition in which a living cell or living cell population is neither increasing or decreasing in total number of cells in a culture. Alternatively, “proliferation” of a cell or population of cells, as the term is used herein, refers to the condition in which the number of living cells increases as a function of time with respect to the original number of cells in the culture.

A “defined culture medium” as the term is used herein refers to a cell culture medium with a known composition.

A “ligand” is a compound that specifically binds with a target receptor.

A “receptor” is a compound that specifically binds to a ligand.

A molecule (e.g., a ligand, a receptor, an antibody, and the like) “specifically binds with” or “is specifically immunoreactive with” another molecule where it binds preferentially with the compound and does not bind in a significant amount to other compounds present in the sample.

By the term “applicator” as the term is used herein, is meant any device including, but not limited to, a hypodermic syringe, a pipette, a bronchoscope, a nebulizer, and the like, for administering a composition of the invention to a mammal.

As used herein, an “instructional material” includes a publication, a recording, a diagram, or any other medium of expression which can be used to communicate the usefulness of a method and/or composition of the invention in a kit for maintaining, proliferating, or administering any composition recited herein. The instructional material of the kit of the invention may, for example, be affixed to a container which contains a composition of the invention or may be shipped together with a container which contains a composition. Alternatively, the instructional material may be shipped separately from the container with the intention that the instructional material and the compound be used cooperatively by the recipient.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

By the term “biologically active antibody fragment” is meant a fragment of an antibody which retains the ability to specifically bind to an SSC epitope.

As the term is used herein, a cell is said to be “eliminated” from a population of cells, or from a culture medium, when the cell no longer exerts one or more of a physical, biological or chemical effect on the population of cells or culture medium. For example, a cell may be eliminated from a culture medium by physically removing the cell using FACS or by using an antibody specific for a cell surface marker unique to that cell. A cell may also be eliminated from a culture medium by rendering the biological activity of that cell inert, such as, for example, by using a neutralizing antibody that is specific for that cell.

A cell is “essentially eliminated” from a population of cells, or from a culture medium, when most, but not all of the total number of such cells no longer exerts one or more of a physical, biological or chemical effect on the population of cells or culture medium. For example, a particular type of cell may be essentially eliminated from a culture medium if at least 75% of the cells of that type are removed from the culture medium by using an antibody specific for a cell surface marker unique to that cell. More preferably, at least 80% of the cells are elminated from the culture medium, even more preferably, at least 85%, more preferably, at least 90%, and even more preferably, at least 95% of the cells are eliminated from the culture medium.

An “inhibitory” cell is a cell that exerts an inhibitory effect on at least one other cell. Inhibitory effects may include, for example, one or more of cell growth inhibition, cell activity inhibition, inhibition of cell maintenance, and inhibition of cell metabolism.

A cell is a “testis-derived” cell, as the term is used herein, if the cell is derived from a testis. By way of a non-limiting example, testis-derived cells include a spermatogonial stem cell, a somatic cell, and a germ cell. Description of the Invention A. Methods of Detecting and Enriching Spermatogonial Stem Cells The present invention features a method of enriching spermatogonial stem cells (SSCs). It has been shown for the first time herein that, using a marker found on an SSC, SSCs can be enriched within a population of cells. It has also been shown for the first time herein that, using a marker found on an SSC, SSCs can be enriched from a population of cells. Stem cell enrichment is useful for various purposes in the field of medical treatment, diagnosis and research, including stem-cell based therapies for repopulation of the cells in an organism, as well as laboratory research to identify growth factors responsible for control of the maintenance and proliferation of stem cells.

In one embodiment of the invention, a method of enriching SSCs in a population of testis-derived cells containing at least one SSCs includes providing an antibody specific for at least one marker expressed on an SSC, contacting the population of cells with the antibody under conditions suitable for formation of an antibody-SSCs complex, and substantially separating the antibody-SSCs complex from the population of cells. In one aspect of the invention, an SSC is a mammalian SSC. In another aspect, an SSC is a mouse SSC. In yet another aspect, an SSC is a human SSC.

As described herein for the first time, SSCs are present in both neonate and adult testis, albeit at a low percentage of total cell population. The present invention has also shown that, in mice, SSCs are present in neonate, pup, and adult testis, and additionally, that SSCs are present in both wild type adult testis and cyrptorchid (i.e., non-descended) adult testis.

This invention therefore provides for the detection, isolation and enrichment of SSCs in a population of testis-derived cells. According to the present invention, an SSC is detected or selected through the binding of a marker, or antigen, found on the cell surface of SSCs, to a reagent that specifically binds the cell surface antigen. As described in detail elsewhere herein, Thy-1 is a marker useful in the methods and compositions of the present invention. This is because it has been shown herein for the first time that Thy-i is found on the cell surface of SSCs that are present in neonate, developing, and adult testis-derived cells. Accordingly, the present invention provides for the detection, isolation and enrichment of SSCs in a population of neonate testis-derived cells. The present invention also provides for the detection, isolation and enrichment of SSCs in a population of adult testis-derived cells. Further, the present invention provides for the detection, isolation and enrichment of SSCs in a population of adult cryptorchid testis-derived cells.

In one embodiment, the present invention provides a method of using Thy-i to enrich SSCs in a population of testis-derived cells. The method of enriching SSCs in a population of testis-derived cells includes providing an antibody specific for Thy-1, contacting the population of cells with the antibody under conditions suitable for formation of an antibody-SSC complex, and substantially separating the antibody-SSC complex from the population of cells, thereby generating an enriched population of SSCs.

The present invention also features a method of enriching SSCs on the basis of SSC cell surface markers other than Thy-1. Other markers useful in the present invention include, but are not limited to, a6-integrin, cahedrin, epithelial glycoprotein-2 (EpCAM), neural cell adhesion molecule (NCAM), glial cell-derived neurotrophic factor family receptor alpha-i (GFRal) and cell adhesion marker CD24 (CD24).

As will be understood by the skilled artisan, any marker that can be displayed on an SSC cell surface can be used in the present invention. In one aspect of the invention, a cell surface marker is a marker that is displayed on the surface of a native SSC. In another aspect, a cell surface marker is a marker that is displayed on the surface of a cell as a result of manipulation of the cell or the marker. In yet another aspect, a marker is one that has been genetically engineered to be expressed on the cell surface.

As will be understood by the skilled artisan, an SSC may be genetically manipulated to express a greater or lesser amount of an existing cell surface marker, or may be genetically engineered to express a heterologous protein or an endogenous protein that is not typically displayed on the SSC cell surface. Techniques and procedures for genetic manipulation of cells to express and display a desired surface marker are generally performed according to conventional methods in the art and various general references (e.g., Sambrook and Russell, 2001, Molecular Cloning, A Laboratory Approach, Cold Spring Harbor Press, Cold Spring Harbor, NY, and Ausubel et al., 2002, Current Protocols in Molecular Biology, John Wiley & Sons, NY), which are provided throughout this document.

In another embodiment of the invention, the marker is treated by chemical modification of a marker typically found on the surface of an SSC. Chemical modification may include contacting the marker with any protein-modifying agent. As will be understood by the skilled artisan, many protein-modifying agents are known in the art, and it will be apparent to the skilled artisan that a protein-modifying agent useful in the present invention may be altered or created de novo, based on the extensive literature surrounding existing agents.

In another embodiment, a marker is treated by enzymatic action. That is, an SSC cell surface marker can be treated by contacting the marker an enzyme, such as a protease, that can modify the marker by proteolytic digestion of all or a portion of the marker. Other enzymes useful for modifying a marker include, but are not limited to, enzymes that can add or remove one or more proteinaceous moieties to a marker, enzymes that can add a non-proteinaceous moiety to, remove a non-proteinaceous moiety from, or alter a non-proteinaceous moiety on a marker (eg., glycosyltransferases, lipases), and enzymes that can alter properties of the amino acid subunits of a protein marker, such as stereochemistry-modifying enzymes.

The invention also features a method of detection of an SSC in a population of testis-derived cells. As described in detail elsewhere herein, SSCs can be positively detected within a population of testis-derived cells by way of the Thy- I surface antigen. In one embodiment of the invention, a method of detecting an SSC in a population of testis-derived cells containing at least one SSC includes providing an antibody specific for Thy-1, contacting the population of cells with the antibody under conditions suitable for formation of an antibody-SSC complex, and detecting the presence of said complex. In one aspect of the invention, an antibody-SSC complex is detected by substantially separating the antibody-SSC complex from the population of cells. As will be understood by the skilled artisan based on the disclosure set forth herein, numerous SSC cell surface moieties, both native and recombinantly engineered, may be used to detect an SSC. Antibodies As will be understood by one skilled in the art, any antibody that can recognize and bind to an SSC marker of interest is useful in the present invention. Methods of making an using such antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, NY). Such techniques include immunizing an animal with a chimeric protein comprising a portion of another protein such as a maltose binding protein or glutathione (GSH) tag polypeptide portion, and/or a moiety such that the marker protein is rendered immunogenic (e.g., a marker protein conjugated with keyhole limpet hemocyanin, KLH) and a portion comprising the respective marker protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the marker protein antigens. Rather, the invention should be construed to include other antibodies, as that term is defined elsewhere herein, to SSC cell surface marker proteins, or portions thereof. Further, the present invention should be construed to encompass antibodies, inter alia, bind to the marker proteins and they are able to bind the marker protein present on Western blots, in solution in enzyme linked immunoassays, in fluorescence activated cells sorting (FACS) assays, in magenetic-actived cell sorting (MACS) assays, and in immunofluorescence microscopy of an SSC transiently transfected with a nucleic acid encoding at least a portion of the marker protein.

One skilled in the art would appreciate, based upon the disclosure provided herein, that the antibody can specifically bind with any portion of the marker protein and the full-length protein can be used to generate antibodies specific therefor. However, the present invention is not limited to using the full-length protein as an immunogen. Rather, the present invention includes using an immunogenic portion of the protein to produce an antibody that specifically binds with a specific SSC cell surface marker protein. That is, the invention includes immunizing an animal using an immunogenic portion, or antigenic determinant, of the cell surface marker protein.

The antibodies can be produced by immunizing an animal such as, but not limited to, a rabbit, a mouse or a camel, with a protein of the invention, or a portion thereof, by immunizing an animal using a protein comprising at least a portion of an SSC cell surface marker protein, or a fusion protein including a tag polypeptide portion comprising, for example, a maltose binding protein tag polypeptide portion, covalently linked with a portion comprising the appropriate amino acid residues. One skilled in the art would appreciate, based upon the disclosure provided herein, that smaller fragments of these proteins can also be used to produce antibodies that specifically bind an SSC cell surface marker protein.

Once armed with the sequence of a specific SSC marker and the detailed analysis localizing the various conserved and non-conserved domains of the protein, the skilled artisan would understand, based upon the disclosure provided herein, how to obtain antibodies specific for the various portions of an SSC marker protein using methods well-known in the art or to be developed.

Further, the skilled artisan, based upon the disclosure provided herein, would appreciate that using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of an SSC marker molecule can be used to produce antibodies that are specific only for that marker and do not cross-react non-specifically with other proteins.

The invention should not be construed as being limited solely to the antibodies disclosed herein or to any particular immunogenic portion of the proteins of the invention. Rather, the invention should be construed to include any other antibodies, as that term is defined elsewhere herein, to Thy-1 or to other SSC marker proteins, such as EpCam, or portions thereof.

The invention encompasses polyclonal, monoclonal, synthetic antibodies, and the like. One skilled in the art would understand, based upon the disclosure provided herein, that the crucial feature of the antibody of the invention is that the antibody bind specifically with an SSC cell surface marker protein. That is, the antibody of the invention recognizes an SSC cell or a fragment thereof (e.g., an immunogenic portion or antigenic determinant thereof), on Western blots, in immunostaining of cells, and immunoprecipitates the marker using standard methods well-known in the art.

One skilled in the art would appreciate, based upon the disclosure provided herein, that the antibodies can be used to immunoprecipitate and/or immuno-affinity purify their cognate antigen as described in detail elsewhere herein, and additionally, by using methods well-known in the art. In addition, the antibody can be used to enrich SSCs in a population of testis-derived cells. Thus, by using an antibody to an SSC cell surface marker, SSCs can be identified, enriched or isolated. One skilled in the art would understand, based upon the disclosure provided herein, that any marker, either native or genetically engineered, expressed on an SSC cell surface, is thus useful in the present invention.

The skilled artisan would appreciate, based upon the disclosure provided herein, that that present invention includes use of either a single antibody recognizing a single SSC marker epitope but that the invention is not limited to use of a single antibody. Instead, the invention encompasses use of at least one antibody where the antibodies can be directed to the same or different SSC marker epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, NY).

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, NY) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., id., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with SSC epitopes. The humanized antibodies of the invention have a human framework and have one or more complementarity determining regions (CDRs) from an antibody, typically a mouse antibody, specifically reactive with SSC. When the antibody used in the invention is humanized, the antibody may be generated as described in Queen, et al. (U.S. Patent No. 6, 180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an SSC epitope, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to a human SSC epitope. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources such as the American Type Culture Collection, Manassas, VA.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies.

Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The use of Old World and New World camelids for the production of antibodies is contemplated in the present invention, as are other methods for the production of camelid antibodies set forth herein.

The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1989, Antibodies: A Laboratory Manual, Cold Spring Harbor, New York). Camelid species for the production of antibodies and sundry other uses are available from various sources, including but not limited to, Camello Fataga S.L. (Gran Canaria, Canary Islands) for Old World camelids, and High Acres Llamas (Fredricksburg, TX) for New World camelids.

The isolation of camelid antibodies from the serum of a camelid species can be performed by many methods well known in the art, including but not limited to ammonium sulfate precipitation, antigen affinity purification, Protein A and Protein G purification, and the like. As an example, a camelid species may be immunized to a desired antigen, for example, an SSC epitope, or fragment thereof, using techniques well known in the art. The whole blood can them be drawn from the camelid and sera can be separated using standard techniques. The sera can then be absorbed onto a Protein G-Sepharose column (Pharmacia, Piscataway, NJ) and washed with appropriate buffers, for example 2OmM phosphate buffer (pH 7.0). The camelid antibody can then be eluted using a variety of techniques well known in the art, for example 0.15M NaCI, 0.58% acetic acid (pH 3.5). The efficiency of the elution and purification of the camelid antibody can be determined by various methods, including SDS-PAGE, Bradford Assays, and the like. The fraction that is not absorbed can be bound to a Protein A-Sepharose column (Pharmacia, Piscataway, NJ) and eluted using, for example, 0.15M NaCl, 0.58% acetic acid (pH 4.5). The skilled artisan will readily understand that the above methods for the isolation and purification of camelid antibodies are exemplary, and other methods for protein isolation are well known in the art and are encompassed in the present invention.

The present invention further contemplates the production of camelid antibodies expressed from nucleic acid. Such methods are well known in the art, and are detailed in, for example U.S. Patents 5,800,988; 5,759,808; 5,840,526, and 6,015,695, which are incorporated herein by reference in their entirety. Briefly, cDNA can be synthesized from camelid spleen mRNA. Isolation of RNA can be performed using multiple methods and compositions, including TRIZOL (Gibco/BRL, La Jolla, Calif.) further, total RNA can be isolated from tissues using the guanidium isothiocyanate method detailed in, for example, Sambrook et al. (1989, Molecular Cloning, A Laboratory Manual, Cold Spring Harbor, NY). Methods for purification of mRNA from total cellular or tissue RNA are well known in the art, and include, for example, oligo-T paramagnetic beads. cDNA synthesis can then be obtained from mRNA using mRNA template, an oligo dT primer and a reverse transcriptase enzyme, available commercially from a variety of sources, including Invitrogen (La Jolla, Calif.). Second strand cDNA can be obtained from mRNA using RNAse H and E. coli DNA polymerase I according to techniques well known in the art.

Identification of cDNA sequences of relevance can be performed by hybridization techniques well known by one of ordinary skill in the art, and include methods such as Southern blotting, RNA protection assays, and the like. Probes to identify variable heavy immunoglobulin chains (VHH) are available commercially and are well known in the art, as detailed in, for example, Sastry et al., (1989, Proc. Nat'l. Acad. Sci. USA, 86:5728). Full-length clones can be produced from cDNA sequences using any techniques well known in the art and detailed in, for example, Sambrook et al. (1989, Molecular Cloning, A Laboratory Manual, Cold Spring Harbor, NY).

The clones can be expressed in any type of expression vector known to the skilled artisan. Further, various expression systems can be used to express the VHH peptides of the present invention, and include, but are not limited to eukaryotic and prokaryotic systems, including bacterial cells, mammalian cells, insect cells, yeast cells, and the like. Such methods for the expression of a protein are well known in the art and are detailed elsewhere herein.

The VHH immunoglobulin proteins isolated from a camelid species or expressed from nucleic acids encoding such proteins can be used directly in the methods of the present invention, or can be further isolated and/or purified using methods disclosed elsewhere herein.

The present invention is not limited to VHH proteins isolated from camelid species, but also includes VHH proteins isolated from other sources such as animals with heavy chain disease (Seligmann et al., 1979, Immunological Rev. 48:145-167, incorporated herein by reference in its entirety). The present invention further comprises variable heavy chain immunoglobulins produced from mice and other mammals, as detailed in Ward et al. (1989, Nature 341:544-546, incorporated herein by reference in its entirety). Briefly, VH genes were isolated from mouse splenic preparations and expressed in E. coli. The present invention encompasses the use of such heavy chain immunoglobulins in the treatment of various autoimmune disorders detailed herein.

As used herein, the term “heavy chain antibody” or “heavy chain antibodies” comprises immunoglobulin molecules derived from camelid species, either by immunization with an peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of VH (variable heavy chain immunoglobulin) genes from an animal.

Once expressed, whole antibodies, dimers derived therefrom, individual light and heavy chains, or other forms of antibodies can be purified according to standard procedures known in the art.

In one embodiment of the invention, a phasge antibody library may be generated. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., supra.

Bacteriophage which encode the desired antibody, may be engineered such that the protein is displayed on the surface thereof in such a manner that it is available for binding to its corresponding binding protein, e.g., the antigen against which the antibody is directed, such as an SSC cell surface marker antigen. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al. (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The MRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105). Methods of Isolation of Antibody-SSC Complexes Once an antibody is bound to an SSC cell surface marker, the complex can be isolated. That is, an antibody-bound SSCs can be substantially separated from a population of testis-derived cells. Methods of identifying or detecting an antibody-antigen complex are well known in the art, and are described in detail elsewhere herein.

Various techniques may be employed to separate the SSCs containing an antibody-bound cell surface marker from cells that do not have an antibody bound cell surface marker by removing antibody-bound SSC cells from the cell mixture. Alternatively, various techniques may be employed to separate the SSCs containing an antibody-bound cell surface marker from cells that do not have an antibody bound cell surface marker by removing from the cell mixture SSC not bound by an antibody.

In one embodiment, the Thy-i SSC cell surface marker is used to separate antibody-bound SSCs from SSCs not conjugated with antibody. In one aspect of the invention, the antibodies may be attached to a solid support to allow for crude separation. The separation techniques employed should maximize the retention of viability of the fraction to be collected. For “relatively crude” separations, that is, separations where up to 10%, usually not more than about 5%, preferably not more than about 1%, of the total cells present having the marker, may remain with the cell population to be retained, various techniques of different efficacy may be employed. The particular technique employed will depend upon efficiency of separation, cytotoxicity of the methodology, ease and speed of performance, and necessity for sophisticated equipment and/or technical skill, all of which is within the ability of the ordinary skilled artisan.

In another embodiment of the invention, the cell surface marker EpCam is used to separate antibody-bound SSCs from SSCs not conjugated with antibody. Therefore, another embodiment of the invention includes an antibody specific for EpCam. EpCam is expressed on SSC from species including, but not 5 limited to rats. It will be understood, based on the disclosure set forth herein, that the novel methods and principles applicable to Thy-1 based separation of SSCs can also be used to separate SSCs using EpCam, with the understanding of the properties and functions of EpCam as known to the skilled artisan.

Procedures for separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., plate, or other convenient technique. Techniques providing accurate separation include fluorescence activated cell sorters, which can have varying degrees of sophistication, e.g., a plurality of color channels, low angle and obtuse light scattering detecting channels, impedance channels, etc., as well as magnetic activated cell sorters.

Conveniently, the antibodies may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the remaining cells. Other techniques include, but are not limited to, dense particles for density centrifugation, an adsorption column, an adsorption membrane, and the like.

In one embodiment of the invention, an antibody specific for an SSC cell surface marker is conjugated to a magnetic bead. A population of testis-derived cells is contacted with the magnetic bead-antibody conjugate, under conditions suitable for binding of the antibody conjugate to an SSC displaying the marker. After incubation under conditions suitable for binding, such as, but not limited to, an incubation at 4° C. for 20 minutes, SSCs positive for the marker are selected by passing the entire sample through a magnetic-based separation apparatus. Upon evacuation of free solution from the apparatus, only the magnetically-retained marker-containing cells will remain. The marker-containing SSC cells are then eluted from the apparatus, resulting in an enriched or purified population of SSC cells. In one aspect of the invention, an SSC marker is Thy-1.

After substantial enrichment of the cells lacking the SSC cell marker, generally by at least about 50%, preferably at least about 70%, the cells may now be separated by a fluorescence activated cell sorter or other methodology having high specificity, such as magnetic activated cell sorting. Multi-color analyses may be employed with the FACS which is particularly convenient. The cells may be separated on the basis of the level of staining for the particular antigens. In a first separation, starting with at least about 1×10¹⁰, preferably at least about 3×10¹⁰ cells, an antibody for Thy-l, for example, may be labeled with one fluorochrome, while the antibodies for other SSC-specific markers may be conjugated to a different fluorochrome. Fluorochromes which may find use in a multi-color analysis include phycobiliproteins, e.g., phycoerythrin and allophycocyanins, fluorescein, Texas red, and the like. The enriched cells may then be further separated by positively selecting for Thy-1, for example. The method should permit the removal to a residual amount of less than about 20%, preferably less than about 5%, of the non-stem cell populations. B. Spermatogonial Stem Cell Culture Systems The present invention also features culture systems for the in vitro maintenance and proliferation of SSCs. In one aspect, an SSC culture system of the invention contains at least one type of serum. In another aspect, as described in greater detail elsewhere herein, an SSC culture system of the invention is serum-free.

In one embodiment, a culture system of the invention is useful for maintaining or culturing SSCs that have been enriched according to methods of the present invention, as set forth in detail elsewhere herein. In another embodiment, a culture system of the invention is useful for maintaining or culturing SSC that have not been previously enriched. By way of a non-limiting example, a population of cells comprising at least one SSC can be cultured using a culture system of the present invention. In one aspect, the culture system can be a serum-free culture system containing one or more components that can specifically or preferentially enable SSC growth and proliferation, but not growth or proliferation of other cells in the population. In this way, a culture system of the present invention can be used to culture SSCs in a population comprising non-enriched SSCs.

A cell culture system useful for the maintenance or proliferation of at least one SSC is described in greater detail in the Experimental Examples set forth herein. Briefly, in an embodiment of the invention, a culture system includes serum-free medium and mitotically inactivated feeder cells (see, for example, Kubota et al., Proc. Natl. Acad. Sci. U.S.A., 97:12132-7 (2000)). As is known to one of skill in the art, feeder cells are cells that are used to supply necessary components for the growth of a cell of interest, such as an SSC. A serum-free medium of the invention can include, but is not limited to, minimum essential medium-alpha (MEMa) or FIO. In one aspect, the medium includes, but is not limited to, one or more of bovine serum albumin, insulin, iron saturated transferrin, free fatty acids, H₂SeO₃, 2-mercaptoethanol, HEPES, putrescine, glutamine, and antibiotics.

In another embodiment, serum supplemented medium of the present invention can be used. In one aspect, serum-containing medium is prepared by adding heat-inactivated FBS to the serum-free medium.

An SSC medium of the invention may also include growth factors. Growth factors useful in the present invention include, but are not limited to, mouse leukemia inhibitory factor (LIF), human insulin-like growth factor-I (IGF-I), human basic fibroblast growth factor (bFGF), mouse epidermal growth factor (EGF), mouse stem cell factor (SCF), and human glial cell line-derived neurotrophic factor (GDNF). Serum-Free Spermatogonial Stem Cell Culture Systems The present invention also features a serum-free culture system for the in vitro maintenance and proliferation of SSCs. This is because it has been shown herein for the first time that SSCs can be maintained and proliferated in the absence of serum in vitro. By way of the present invention, a culture system having minimal, defined conditions has been established for the in vitro culturing of SSCs, which system provides the ability investigate SSC biology in a defined way, as well as the ability to identify individual factors required for maintenance and expansion of SSCs.

In one embodiment of the invention, a serum-free SSC culture system includes a serum-free defined medium and mitotically inactivated feeder cells. In one aspect of the invention, the feeder cells are STO cells. In another aspect of the invention, the feeder cells can include, but are not limited to, fibroblasts, including mouse embryonic fibroblasts, kidney epithelial cells, and vascular endothelial cells.

In one aspect of the invention, the serum-free defined medium includes minimal essential medium-a (MEMo). In another aspect of the invention, the serum-free defined medium includes Ham's FIO culture medium. In yet another aspect of the invention, the serum-free defined medium can include, but is not limited to, RPMI bicarbonate-buffered medium and Dulbecco's MEM : Ham's Nutrient Mixture F-12 (DMEM/F 12). As will be understood by the skilled artisan when armed with the present disclosure, a serum-free defined medium of the present invention also includes a mixture of two or more media.

Therefore, the present invention includes a composition including a defined medium and a mitotically inactivated feeder cell for the maintenance or proliferation of SSCs. As will be understood based on the disclosure set forth herein, a serum-free culture system of the invention is useful for the maintenance or expansion of SSCs. In one aspect of the invention, SSCs useful in the culture system are SSCs enriched using the methods or compositions of the present invention. In another aspect, SSCs useful in the culture system are SSCs that have not been previously enriched according to the methods or compositions of the present invention.

In one embodiment of the invention, a serum-free defined medium further includes SSCs. The skilled artisan will understand, in view of the present disclosure, that SSCs from any source may be maintained or expanded using the serum-free culture system of the invention. That is, SSCs obtained from a population of testis-derived cells can be obtained from testis cells derived from any mammalian source including, but not limited to, human testis and mouse testis. Sources of SSCs further include wild type adult testis, adult testis having one or more genetic mutations, juvenille testis, neonate testis, and cryptorchid adult testis. Methods for introduction of genetic mutations to the DNA in a cell, such as an SSC, are well-known in the art and will not be discussed further herein.

It will also be understood, based on the disclosure set forth herein, that a sterile male can be a source for SSCs. Therefore, another aspect of the invention includes a sterile male as a source of SSCs of the present invention.

As described in detail elsewhere herein, the basic serum-free defined medium of the present invention may further include any components known by the skilled artisan to be useful in the culturing of cells. In one embodiment, a serum-free defined medium includes at least one growth factor. Growth factors useful in the present invention include, but are not limited to, stem cell factor (including mouse SCF), glial cell line-derived neurotrophic factor (GDNF), GDNF-family receptor (including GFRaI), leukemia inhibitory factor (LIF), basic fibroblast growth factor (including human bFGF), acidic fibroblast growth factor (aFGF), epidermal growth factor (EGF), insulin-like growth factor (including IGF-I), platelet-derived growth factor (PDGF), and transforming growth factor (including TGF-βI through III, as well as the TGF-P superfamily BMP-1 through 12, GDF 1 through 8, dpp, 60A, BIP, OF).

The present invention therefore also includes methods of maintaining or proliferating SSCs in a serum-free defined culture medium. In one embodiment, the invention features a method of maintaining SSCs in a serum-free culture system. The method includes providing at least one SSC in a serum-free culture system as defined in detail elsewhere herein.

An SSC may be identified as being “maintained” in the serum-free defined culture system by assessing the activity of an SSC at various time points in the culture medium and comparing the activity with the activity of the SSCs at the start of the culture period. As will be understood by the skilled artisan, little or no loss of activity is an indication that SSCs have been maintained in culture. Methods of measuring. the activity of SSCs are described in detail elsewhere herein.

In another embodiment, the invention features a method of proliferating SSCs in a serum-free culture system. The method includes providing at least one SSC in a serum-free culture system as defined in detail elsewhere herein. A SSC may be identified as being “proliferated” in the serum-free defined culture system by assessing the SSC activity at various time points in the culturing process and comparing the activity with the activity of the SSC at the start of the culture period. An increase in the activity between the start of the culture period and any later time point is an indication that SSCs have been proliferated. Methods of measuring the activity of SSCs are described in detail elsewhere herein.

Additionally, the degree of proliferation of SSCs in a serum-free culture system of the present invention may be assessed by counting the number of cells present at a specific point in time during SSC cell culture and comparing the value to the number of cells present at the start of the culture period. Based on the disclosure set forth herein, the skilled artisan will understand that these and other methods of assessing SSC maintenance and proliferation may be used. These methods include, but are not limited to, FACS and MACS. C. Methods of using and determining the effect of a growth factor on SSCs The present invention further includes a method of determining the effect of a growth factor on an SSC. In one embodiment, the method uses a serum-free SSC culture system, wherein the culture system includes a first population of enriched SSCs, serum-free defined culture medium, and a population of mitotically inactivated STO feeder cells. The culture system is contacted with at least one growth factor, and the activity of said first population of enriched SSCs is assessed. The assessed activity of the first population of enriched SSCs is compared with the activity of a second population of enriched SSCs, wherein the second population of said SSCs is cultured in a growth factor-free culture system that is otherwise identical to the culture system used in conjunction with the first population of enriched SSCs.

In one aspect of the invention, a higher level of SSC activity in the population of first enriched SSCs is an indication that the growth factor or mixture of growth factors enhances the activity of an SSC. Alternatively, in another aspect of the invention, a lower level of SSC activity in the population of first enriched SSCs is an indication that the growth factor or mixture of growth factors inhibits the activity of an SSC. Methods of measuring the activity of SSCs are described in detail elsewhere herein.

As will be understood by the skilled artisan, any growth factor, from any source, can be used in the present invention. This is because a method of the present invention will provide information regarding the effect, or absence thereof, of any growth factor on an SSC in a cell culture system of the invention. Growth factors useful in the present invention include, but are not limited to, stem cell factor (including mouse SCF), glial cell line-derived neurotrophic factor (GDNF), GDNF-family receptor (including GFRal), leukemia inhibitory factor (LIF), basic fibroblast growth factor (including human bFGF), acidic fibroblast growth factor (aFGF), epidermal growth factor (EGF), insulin-like growth factor (including IGF-I), platelet-derived growth factor (PDGF), and transforming growth factor (including TGF-P I through III, as well as the TGF-P superfamily BMP-I through 12, GDF 1 through 8, dpp, 60A, BIP, OF).

Methods of identifying an increase or decrease in a biological process of an SSC that can be modulated using a growth factor according to the present invention are set forth in detail elsewhere herein. Additional methods useful for identifying an increase or a decrease in a biological process will be apparent to the skilled artisan in view of the present disclosure, and will therefore not be discussed in detail.

The present invention also features a method of using at least one signaling pathway of a growth factor receptor to impart the effect of a growth factor on an SSC. That is, one or more cell signaling pathways typically associated with stimulation of a cell surface receptor can be used to impart the effect of growth factor binding to the cell surface receptor. In one embodiment of the invention, as set forth above, a signaling pathway can be stimulated by way of growth factor interaction with one or more growth factor receptors. In one aspect, the growth factor is a growth factor typically associated with the receptor. In another embodiment, the growth factor is a growth factor not typically associated with the receptor. In yet another embodiment of the invention, the growth facor receptor is stimulated by a non-growth factor molecule.

In an embodiment of the invention, SSC activity can be modulated through one or more GDNF signaling pathways. In one aspect of the invention, SSC activity is modulated through a GDNF signaling pathway by a method comprising contacting an SSC with GDNF. In another aspect, SSC activity is modulated through a GDNF signaling pathway by a method comprising contacting an SSC with a non-GDNF component or modulator of a GDNF signaling pathway.

Cellular responses to GDNF are mediated by a multicomponent receptor complex consisting of c-Ret receptor tyrosine kinase and a glycosyl phosphatidylinositol-anchored ligand-binding subunit, GFRal, in many cell types (Airaksinen et al., Nat. Rev. Neurosci., 3, 383 (2002)). Mouse spermatogonia express GFRal (Meng et al., Science, 287, 1489 (2000)), and adding soluble GFR(l molecules to a cell culture can potentiate the stimulatory signal through the c-Ret receptor or modulate the signaling pathways (Paratcha et al., Neuron, 29, 171 (2001)). Therefore, in another embodiment of the present invention, basic fibroblast growth factor (bFGF), a critical growth factor for primordial germ cells (PGCs) in vitro, affects the GDNF signaling pathway. As described in greater detail elsewhere herein, soluble GFRa I along with GDNF improves the maintenance of germ cell clumps and promotes expansion of SSCs in culture. Further, a similar response is obtained when bFGF is added to a culture in conjunction with GDNF, as described in detail elsewhere herein for the first time.

Therefore, in an aspect of the invention, SSC activity is modulated through a GDNF signaling pathway by a method comprising contacting an SSC with a non-GDNF component or modulator of a GDNF signaling pathway. Such components and/or modulators include, but are not limited to, c-Ret, c-Ret receptor tyrosine kinase, GFRa I, bFGF, NCAM, Oct-4, and molecules involved in signal transduction of GDNF, among others. D. Transplantation of Spermatogonial Stem Cells In one aspect of the invention, one or more SSCs can be transplanted into a recipient testis. Transplantation methods are generally known in the art, and will not be discussed in extensive detail herein. For general cell transplantation methods involving the testis, see Kanatsu-Shinohara et al. (PNAS, 99:1383-1388 (2002)), Brinster (I) (U.S. Patent No. 6,215,039), and Brinster (II) (U.S. Patent No. 5,858,354), all of which are hereby incorporated by reference herein in their entirety. Brinster (I) and (II) demonstrate, in part, that SSCs transplanted from a donor to an immunologically tolerant mouse or other compatible recipient will replicate and be maintained in the recipient.

In an embodiment of the invention, one or more SSCs are introduced into the tubules of a testis. For example, a recipient male mammal can be anesthetized and the testis (or testes) surgically exposed. In one embodiment, using micromanipulation methods, a thin glass needle is introduced into exposed tubules, one after another, and each tubule is injected with a solution containing the primitive cells being used to colonize the tubule. In another embodiment, one or more SSCs can also be introduced by injecting them into other parts of the tubular system, e.g. the lumen of the rete testes. As will be understood by the skilled artisan, injection methods are available that minimize the number of injection sites and increase the efficiency of injection of SSCs into a recipient male.

A cell suspension of one or more SSCs for injection can comprises an injection medium and at least one SSC at a suitable concentration. By way of a non-limiting example, the injection medium can comprise one or more of NaCl, Na₂HPO₄, KCl, KH₂PO₄, EDTA, pyruvate, lactate, glutamine, glucose, bovine serum albumin, and DNAse I. The pH of the injection media is suitably in the range of 7.0-7.7, but as will be understood by the skilled artisan, can be adjusted to be more basic or more acidic depending upon the medium composition, the cell type and/or concentration, and the microenvironment of the recipient injection site.

In another embodiment of the invention, other systems can be used for the introduction of one or more SSCs into a recipient male. These include injection into the vas deferens and epididymis or manipulations on fetal or juvenile testes, techniques to sever the seminiferous tubules inside the testicular covering, with minimal trauma, which allow injected cells to enter the cut ends of the tubules. Alternatively neonatal testis (or testes), which are still undergoing development, can be used.

As set forth elsewhere herein, SSCs entering the testicular tubule are generally protected from destruction by the immunologically privileged environment of the internal lumen of the tubule. Cells that leak from the tubule are typically destroyed by the immune system of the host since the cells are foreign to the animal.

In another embodiment of the invention, animal strains are used which are from different species to provide donor cells (xenogeneic transfer). Sources of SSCs include, but are not limited to human, rodent, including rat and mouse, primate, including baboon, cow and dog.

The present invention is applicable to any species of animals, including humans, in which the male has testes, including but not limited to non-human transgenic animals. The invention is also not limited to mammalian species. It can be used to. provide animals and animal lines of many types with a single, or many, novel genetic modification(s) or novel characteristic(s). The animals to which the present invention can be applied include humans, non-human primates (eg., monkeys, baboons), laboratory animals, such as rodents (eg., mice, rats, etc.), companion animals (eg., dogs, cats), birds (such as chickens and turkeys), wild animals (eg., buffalo, wolves), endangered animals (eg., elephants, leopards), and zoo species (such as tigers, zebras, lions, pandas, giraffes, polar bears, monkeys, sea otters, etc.) which can be modified to permit their use in cellular diagnosis or assays. The present invention may also be advantageously applied to farm animals, including domesticated ruminants and fowl (e.g., cattle, chickens, turkeys, horses, swine, etc.), to imbue these animals with advantageous genetic modification(s) or characteristic(s).

In another embodiment of the invention, the donor and recipient mammal can be the same mammal. In one aspect, a population of cells comprising SSCs are collected from a mammal prior to destruction of the germ cell population and then reintroduced thereafter. This embodiment would preserve the ability of the mammal to reproduce following radiation therapy, for example which may be necessary during the treatment of cancer. Alternatively, spermatogonial stem cells may be harvested from the mammal and kept in culture or frozen. In this aspect of the invention, when progeny are desired, the stem cells are transplanted to a recipient mouse testis. The donor mammal egg can then be fertilized by spermatozoa developed in the recipient mouse testis. There are no time constraints on this procedure since the stem cells continually undergo self-renewal.

Once an initial fertilization event is achieved and the resulting offspring is fertile, the mammal line with its novel genetic modification or. characteristic is established where the novel genetic modification or characteristic is present in both male and female offspring. Thus, in accordance with the invention, a mammal may be produced harboring, in its testes only, a biologically functional germ cell which is not native to that mammal by repopulating its testicular seminiferous tubules. This (parent) mammal can produce progeny. Every cell in the progeny is genetically non-native as compared to the parent mammal.

Both the parent mammal and its progeny provided by the present invention have multiple and varied uses, including, but not limited to, uses in agriculture and biomedicine, including human gene therapy. An illustrative agricultural use of the present invention relates to increasing the breeding potential of a valuable stud animal. In another aspect of the present invention, chimeric animals useful in either biomedicine or agriculture are provided. As will be understood by the skilled artisan, when armed with the present application, the present invention provides an advantageous complementation to existing transgenic animal techniques.

The present invention alleviates the present difficulty and expense of embryological transgenic work. In one embodiment of the present invention, spermatogonial stem cells can be genetically modified and then transferred to recipient testes. The valuable genetic traits present in the resultant germ cells can be passed onto the (transgenic) progeny of the recipient stud. This particular application of the present invention is important for the genetic engineering of large agricultural animals.

As set forth herein, the present invention also has applications in gene therapy, including human gene therapy. By way of a non-limiting example, a patient with a deleterious genetic trait could undergo a testicular biopsy. Isolated stem cells can be genetically modified to correct the deleterious trait. The patient then undergoes a treatment to remove the remaining germ cells from his testes, for example by specific irradiation of the testes. His testes (now devoid of germ cells) can then be recolonized by his own, genetically-corrected, stem cells. The patient can then father progeny free from the worry that he would pass on a genetic disease to his progeny. Alternatively, the stem cells with the corrected gene can be transplanted to a mouse and the resulting sperm used for fertilizing eggs, thereby foregoing the need for reimplanting stem cells into the original human testis. E. Kits The invention includes various kits which comprise a serum free culture system for the maintenance or proliferation of at least one SSC. Although exemplary kits are described below, the contents of other useful kits will be apparent to the skilled artisan in light of the present disclosure. Each of these kits is included within the invention.

In one aspect, the invention features a kit for maintaining at least one SSC in a serum-free culture system, comprising a culture system comprising serum-free defined culture medium and mitotically-inactivated STO feeder cells, an applicator, and instructional material, wherein the instructional material comprises instructions for the use of the kit to maintain at least one SSC in the serum-free culture system. In another aspect, the invention features a kit for proliferating at least one SSC in a serum-free culture system, comprisin a culture system comprising serum-free defined culture medium and mitotically-inactivated STO feeder cells, an applicator, and instructional material, wherein the instructional material comprises instructions for the use of the kit to proliferate at least one SSC in the serum-free culture system.

The invention also features a kit for administering a population of enriched SSCs to a mammal, comprising a culture system comprising serum-free defined culture medium and mitotically-inactivated STO feeder cells, an applicator, and instructional material, wherein the instructional material includes instructions for the use of the kit to proliferate at least one SSC in the serum-free culture system and for the applicator-based administration of the enriched SSCs to a mammal.

The particular applicator included in the kit will depend on, e.g., the method and/or the composition used to introduce a population of enriched SSCs to a cell. Such applicators are well-known in the art and may include, among other things, a membrane, an implant, a syringe, and the like. Moreover, the kit comprises an instructional material for the use of the kit. These instructions simply embody the disclosure provided herein.

The kit may also include a pharmaceutically-acceptable carrier. The composition is provided in an appropriate amount as set forth elsewhere herein. Further, the route of administration includes, but should not be limited to, direct contact with the desired site of administration, as well as contact with a cell or tissue adjacent to the desired site of administration.

Compositions and methods for the isolation, purification, enrichment, proliferation and maintenance of SSC, as encompassed by the kits of the invention, are described in detail elsewhere herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Experimental Example 1

Enrichment of Spermatogonial Stem Cells using Thy-1 and Establishment of Non-Serum Culture Conditions for Spermatogonial Stem Cells Donor Mice and Cell Collection

Cryptorchid and wild type adult donor testis cells were obtained from the transgenic mouse line B6.129S7-Gtrosa26 (designated ROSA; The Jackson Laboratory, Bar Habor, ME) that expresses the Escherichia coli LacZ gene in virtually all cell types, including all stages of spermatogenesis (Nagano et al., APMIS, 106:47-55 (1998)). Neonate (0.5-1.5 days postpartum, dpp; day of birth is 0.5 dpp), and pup (4.5-5.5 dpp) testis cells were collected from the hemizygous transgenic mice, C57BL/6 x ROSA FI hybrid. Experimental cryptorchid testes were produced as previously described (Shinohara et al., Dev. Biol., 220:401-11 (2000)). Cell suspensions from cryptorchid adult, wild type adult, neonate, and pup testes were prepared by enzymatic digestion (Ogawa et al., Int. J. Dev. Biol., 41:111-22 (1997)). In several experiments, testis cells were fractionated by using Percoll (Sigma, St. Louis, MO) to remove cellular debris and large cells. The dissociated testis cell suspension, after enzymatic digestion, was overlaid on 30% (v/v) Percoll prepared in Dulbecco PBS containing 1% FBS (Hyclone, Logan, UT) and centrifuged at 600g for 8 mm at 4° C. Cells from the interphase and Percoll phase were collected as top fraction. Sedimented cells were used as bottom fraction. Cell Staining and Fluorescence-Activated Cell Sorting 6 Dissociated testis cells were suspended (5 x 10 cells/ml) in Dulbecco PBS supplemented with 1% FBS, 10 mM HEPES (Sigma, St. Louis, MO), 1 mM pyruvate (Sigma, St. Louis, MO), antibiotics (50 units/ml penicillin and 50,ug/ml streptomycin; Invitrogen; Carlsbad, Calif.), and 1 mg/ml glucose (Sigma, St. Louis, MO) (PBS-S). All antibodies were obtained from BD Biosceiences (Franklin Lakes, NJ), unless otherwise stated. For fluorescence-activated cell sorting (FACS) analysis of cryptorchid adult testis cells, dissociated cells were incubated with anti-12 microglobulin (p2M; S19.8) and with biotin-conjugated anti-Thy-i (53-2.1) antibodies for 20 minutes on ice and washed twice with excess PBS-S. All staining and washing were performed with a similar protocol. Then, cells were stained with Alexa Fluor 647-conjugated goat anti-mouse lgG2b antibody (Alexa Fluor 647-lgG2b; Molecular Probes, Eugene, OR) and Alexa Fluor 488-conjugated streptavidin (Alexa Fluor 488-SAv; Molecular Probes) for P2M and Thy-1, respectively. For FACS analysis of wild type adult testis cells, cell suspensions from the bottom fraction after Percoll centrifugation were stained with anti-a6-integrin (GoH3), anti- 02M, and R-phycoerythrin (PE)-conjugated anti-Thy-1 (30H- 12) antibodies. The staining pattern of mouse testis cells by two anti-Thy-1 antibodies (53-2.1 and 30H-12) showed no difference. a6-integrin was detected by Alexa Fluor 488-SAv after staining with biotin-conjugated rat anti-mouse IgG,/,a (G28-5) antibody. P2M was detected by Alexa Fluor ⁶⁴⁷-IgG2b. For FACS analysis of pup and neonate testis cells, dissociated cells were stained with anti- 02M, biotin-conjugated anti-Thy-1 (53-2.1), and PE-conjugated anti-av-integrin (RMV-7) antibodies, followed by Alexa Fluor ⁶⁴⁷-IgG2b and Alexa Fluor 488-SAv. Prior to FACS, 1 gg/ml propidium iodide (Sigma, St. Louis, MO) was added to the cell suspensions to exclude dead cells. Cell sorting was performed on a FACStar Plus (BD Biosciences) equipped with Coherent Enterprise II laser (488 mn) and air-cooled helium neon laser (633 mn) and operated by Flow Cytometry and Cell Sorting Shared Resource at the University of Pennsylvania. Cells were sorted into 5-ml polypropylene tubes containing 4 ml of PBS supplemented with 10% FBS, 10 mM HEPES, 1 mM pyruvate, antibiotics, and 1 mg/ml glucose. An aliquot of stained cells in each experiment was not used for FACS (unsorted control). The sorted and unsorted control cells were centrifuged and resuspended in 3 ml of Ham's Nutrient Mixture F- 10 (FlO) supplemented with 10% FBS, 50 gM 2-mercaptoethanol (Sigma, St. Louis, Mo.), 10 mM HEPES, 2 mM glutamine (Invitrogen; Carlsbad, Calif.) and antibiotics. The tubes were gassed with 5% C0₂ and stored overnight at 4° C. (Kubota et al., PNAS., 100:6487-6492 (2003)). Magnetic-Activated Cell Sorting Magnetic microbeads conjugated to anti-Thy-1 antibody (30-H1 2; Miltenyi Biotec, Gladbach, Germany) were used for magnetic-activated cell sorting (MACS) to enrich Thy-1+cells from testis cell suspensions. The procedure to obtain Thy-1+cells was performed according to the manufacture's protocol with minor modification. Briefly, dissociated cells from cryptorchid adult, wild type adult, pup, or neonate testes were fractionated by Percoll centrifugation as described earlier. The single cell suspension (3-8 x 106 cells in 90 μl of PBS-S) from the bottom fraction of Percoll centrifugation was incubated with 10 μl of Thy-1 microbeads for 20 minutes at 4° C. After rinsing with PBS-S, Thy-1+cells were selected by passing through an MS separation column (Miltenyi Biotec, Cologne, Germany) that was placed in a magnetic field. After removal of the column from the magnetic field, the magnetically retained Thy-1+ cells were eluted. Cell Culture The basic culture system consisted of serum-free medium and mitotically inactivated STO cell feeders as described previously for hepatic progenitors with minor modification (Kubota et al., Proc. Natl. Acad. Sci. U.S.A., 97:12132-7 (2000)). Feeder cells are cells that are used to supply necessary components for the growth of a cell of interest, such as an SSC. “Compoenents” supplied by feeder cells include one or more of cell-cell contact, tropic factors, and the like. The serum-free medium for SSCs consisted of minimum essential medium-alpha (MEMA; Invitrogen; Carlsbad, Calif.) or FIO to which was added 0.2% bovine serum albumin (ICN Biomedicala, Irvine, Calif.), 5 μg/ml insulin (Sigma, St. Louis, MO), 10 μg/ml iron saturated transferrin (Sigma, St. Louis, MO), 7.6 eq/l free fatty acids (Chessebeuf et al., In Vitro, 20:780-95 (1984)), 3×10⁻⁸ M H₂SeO₃ (Sigma, St. Louis, MO), 50 μM 2-mercaptoethanol, 10 mM HEPES, 60 /M Putrescine (Sigma, St. Louis, MO), 2 mM glutamine, and antibiotics. Free fatty acids comprised palmitic, palmitoleic, stearic, oleic, linoleic, and linolenic acids in the respective millmolar proportions of 31.0 : 2.8: 11 .6: 13.4: 35.6: 5.6 for 100 meq/l stock solution (Chessebeuf et al., In Vitro, 20:780-95 (1984)). Serum supplemented medium was prepared by adding heat inactivated (56° C., 30 minutes) FBS into the serum-free medium at the concentrations indicated (0.1-10%; v/v). Growth factors used were mouse leukemia inhibitory factor (LIF; Chemicon International, Temecula, Calif.), human insulin-like growth factor-I (IGF-I; R&D systems, Minneapolis, MN), human basic fibroblast growth factor (bFGF; BD Biosceinces), mouse epidermal growth factor (EGF; BD Bloscicences), mouse stem cell factor (SCF; R&D systems), and human glial cell line-derived neurotrophic factor (GDNF; R&D systems).

For cultures with P2M-Thy-1+cells isolated by FACS from ROSA cryptorchid adult testis cells, 2 x 104 02M Thy-1+cells were plated in two wells of a 6-well plate (1 x 104 cells/9.6 cm²) with STO feeders or newborn (NB) testis cell feeders and in serum-free media or in serum-supplemented media. STO cells (STO SNL76/7 cells) and STO feeders were prepared as described (Robertson, Oxford, England: IRS Press, 71-112 (1987)). Briefly, STO cells were maintained in Dulbecco modified Eagle medium (DMEM) supplemented with 7% FBS, 100 μM 2-mercaptoethanol, 2 mM glutamine, and antibiotics. For making feeders, STO cells were treated with 10 g/ml of mitomicin C (Sigma, St. Louis, MO) for 3-4 hours and plated at a density of 5 x 10⁵ cells per well of a 6-well plate coated with 0.1 % gelatin (Sigma, St. Louis, MO) in the same medium. Before culture with donor testis cells, STO feeders were rinsed with Hank Balanced Salt Solution twice. For NB testis feeders, NB testis cells (0.5-1.5 dpp) from C57BL/6 x 129/SvCP Fl hybrid mice were prepared by enzymatic digestion. Two to 2.5 x 106 cells were placed in a 10 cm tissue culture dish and cultured in FIO supplemented with 10% FBS, 50 RM 2-mercaptoethanol, 10 mM HEPES, 2 mM glutamine and antibiotics for 2 days. Mitomycin C treated-NB testis feeders were prepared as described for STO feeders. FACS-sorted P2M Thy-l+cells were maintained on feeders for 8-10 days. Culture medium (2 ml/well) was changed every other day. Likewise, Thy-1 microbead-selected cells were cultured on STO feeders in the MEMA based medium in two wells of a 6-well plate at a density of 1 x 104 cells per well (1 x 104 cells/9.6 cm²). When the culture period was extended to 2 weeks, cells were harvested from two wells after one week of culture and reseeded on fresh STO feeders in 2 wells of a 6 well-plate. All cultures were maintained at 37° C. in a humidified 5 % C0₂ atmosphere. Testis Cell Transplantation and Analysis of Recipient Testes C57BL/6 x 129/SvCP Fl hybrid recipient male mice were used as recipients. The mice were treated with busulfan (55 mg/kg, Sigma) at 5-7 weeks of age to deplete endogenous germ cells in the testes (Brinster et al. Proc. Natl. Acad. Sci. U.S.A., 19:11298-302 (1993), Brinster et al., Proc. Natl. Acad. Sci. U.S.A., 91:11303-7 (1994)). Approximately 10 il of donor cell suspension was transplanted into the seminiferous tubules of each recipient testis through the efferent duct 4-6 weeks after busulfan treatment (Ogawa et al., Int. J. Dev. Biol., 41:111-22 (1997)). The injection resulted in 70-80% filling of the tubules in each testis. Preparation of donor cells isolated by FACS for transplantation was described previously (Kubota et al., PNAS., 100:6487-6492 (2003)). Thy-1+cells obtained by MACS were resuspended in MEMa-based serum-free medium and transplanted into the recipient testes. Cultured donor cell suspensions for transplantation were prepared in the same medium as used for each culture condition. The institutional Animal Care and Use Committee of the University of Pennsylvania approved all experimental procedures in accordance with the Guide for Care and Use of Laboratory Animals from the National Academy of Sciences.

Two months after transplantation, recipient testes were collected and stained with 5-bromo-4-chloro-3-indolyl 0 -D-galactoside (X-gal) to visualize donor-derived spermatogenic colonies (Nagano et al., Biol. Reprod., 60:1429-36 (1999)). Individual blue stretches of spermatogenesis represent spermatogenic colonies generated from donor-derived single SSCs (Nagano et al., Biol. Reprod., 60:1429-36 (1999), Dobrinski et al., Mol. Reprod. Dev., 53:142-8 (1999)). Colony number was counted by using a dissection microscope. For the analysis of testes injected with testis cells isolated by FACS or MACS, colony number was normalized to 10⁵ cells injected because the number of cells that could be recovered and injected varied. Colony number for injected cultured testis cells was normalized to 10⁵ cells originally seeded in culture to compare with values of colony number generated by the cell population prior to culture. Statistical analyses were performed by analysis of variance (ANOVA) using SAS version 8.2 (SAS institute, Cary, N.C.). Thy-1 is a surface marker for spermatogonial stem cells in wild type adult testes Surface expression of Thy-I in wild type adult testis cells as well as cryptorchid adult testis cells was analyzed to determine whether Thy-I is a positive marker of SSCs throughout postnatal life of the mouse. Initially, cryptorchid testis cells were stained with 02M, the light chain of MHC-l, and Thy-I antibodies and analyzed by FACS (FIG. 1A, B). Some P2M+cells produced auto-fluorescent signals in the control sample that was stained with 02M antibody only (FIG. 1A; gate 3, G3); however, the Thy-I+cell population was identified clearly in the 02M cells (FIG. 1B, G1). Three populations, 02M-Thy-l+(GI), 02M-Thy-l- (G2), and 02M+cells (G3), were isolated by FACS and transplanted into the seminiferous tubules of busulfan-treated infertile recipient mice to determine the stem cell activity in each fraction. Two months after transplantation, spermatogenic colonies in the recipient testes stained with X-gal were counted (FIGS. 1C and 1D). Stem cell activity was detected almost exclusively in the 132M-Thy-l+cell fraction while few spermatogenic colonies were generated from 02M-Thy-i- cells and 02M+cells, indicating that 02M-Thy-l+cells contained most (-95%) of SSCs in the testis. The ,2M-Thy-1+cells produced about 280 colonies of spermatogenesis per 105 cells transplanted (FIG. 1C), and stem cell concentration was enriched 15-fold (282.6/18.6) over unsorted cryptorchid testis cells.

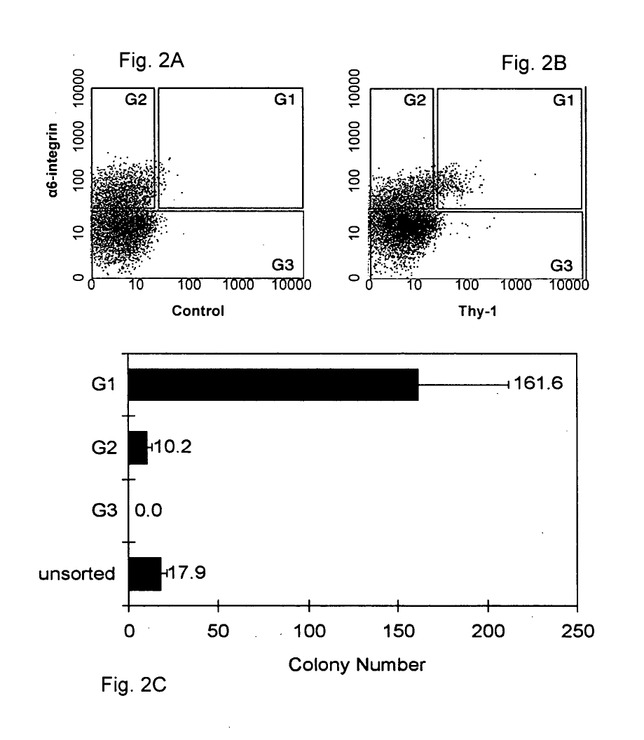

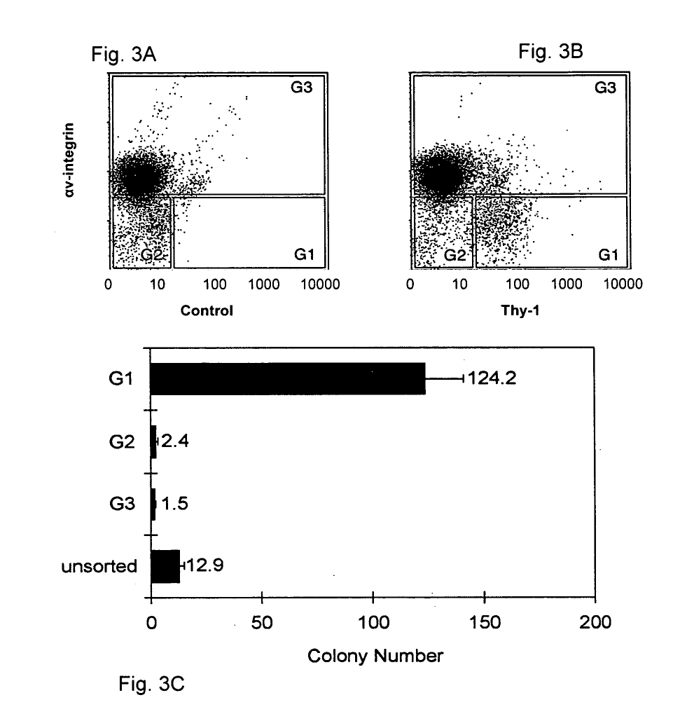

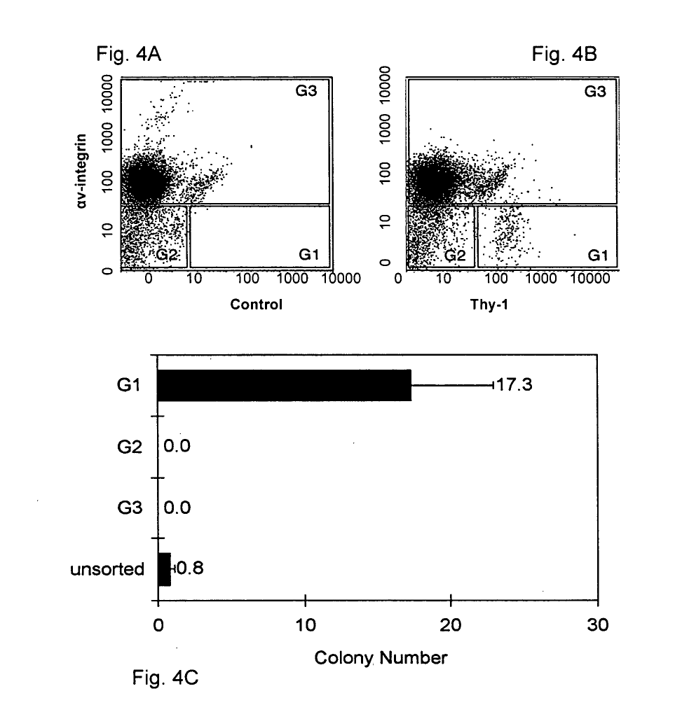

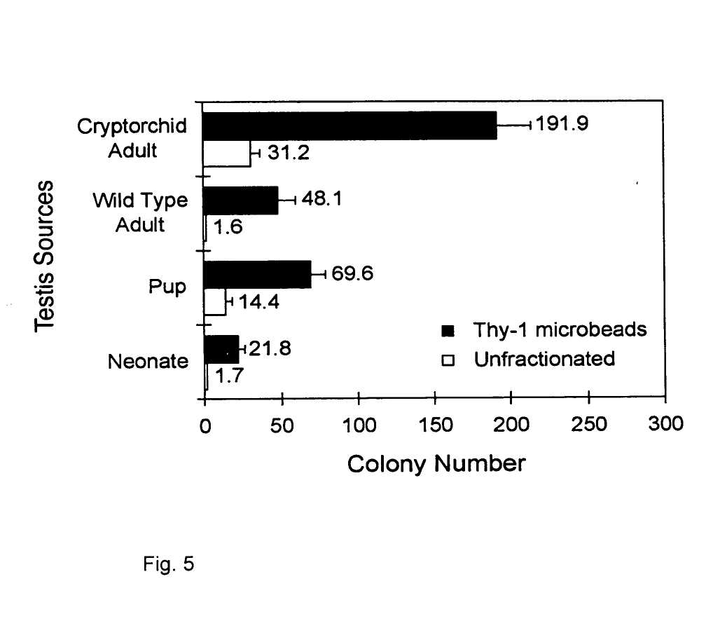

Subsequently, Thy-I expression of wild type adult testis cells was analyzed. FACS analysis, however, did not show a distinct Thy-l+subpopulation in the wild type adult testis probably due to the very low number of Thy-1+cells among the many differentiating germ cells, auto-fluorescent large cells, and cellular debris. Therefore, cell separation was accomplished by centrifugation in Percoll to concentrate SSCs and to reduce cellular debris before antibody staining for FACS analysis. Approximately 11% of the original testis cells were sedimented in the bottom of the centrifugation tube after Percoll separation. All floating cells (top fraction) including the Percoll phase were also collected. The top fraction contained about 68% of total cells, indicating that about 79% of cells were recovered after Percoll centrifugation. To examine the stem cell activity of each fraction, a transplantation assay was performed. The number of spermatogenic colonies in the bottom fraction and the top fraction were 12 and 1.4 per 105 cells transplanted, respectively, while the original wild type testis cells generated 2.6 colonies per 105 transplanted. This result indicated that the bottom fraction contained about a 5-fold enriched population of SSCs compared to the original wild type adult testis cells. The bottom fraction was stained with antibodies against Thy- 1, 02M and a 6-integrin, a surface marker of SSCs [(Shinohara et al., Proc. Natl. Acad. Sci. U.S.A., 96:5504-9 (1999)]. P2M+cells, which were about 10% in the bottom fraction, were gated out for analysis of Thy- I and a6-integrin expression. In addition, side scattering cells were removed by preliminary gating because of autofluorescence. In the 02M cells, FACS analysis identified Thy-1+cells in the a6-integrin+cell fraction (FIG. 2B). About 10% of the a6-integrin+cells expressed Thy-i (2.6%/25%; FIG. 2 legend). Three subpopulations, Thy-1+a6-integrin+(G1), Thy-1- a6- integrin+(G2), and a6-integrin (G3), were isolated by FACS followed by transplantation. After two months, recipient testes were analyzed. The spermatogenic colony number in Thy-1+a6-integrin+, Thy-lF a6-integrin+, and a6-integrin- cells, were 162, 10, and 0 per 105 cells transplanted, respectively (FIG. 2C). These results confirmed that Thy-I antigen is expressed on SSCs in wild type adult testis as well as cryptorchid adult testis cells. However, the concentration of SSCs in the P2MThy-l+cryptorchid testis cells was higher than that in the 02M- Thy-1+a6-integrin+wild type testis cells. Thy-I is expressed on spermatogonial stem cells in PuP and neonate testes Thy-i expression on SSCs in pup and neonate testes was examined. Preliminary experiments showed that there were few P2M+cells in pup and neonatal testis cells. Therefore, the testis cell suspension was stained with anti-av-integrin antibody as well as anti-p2M and anti-Thy-I antibodies, because most of PM+cells expressed av-integrin in the cryptorchid testis cells, and SSCs do not express av-integrin in the adults (Kubota et al., PNAS., 100:6487-6492 (2003)). FACS analysis of the stained testis cells identified Thy-I+cells in the av-integrin population in both pup and neonate testis cells (FIGS. 3 and 4). In pups, the staining pattern of (Xv-integrin in the Thy-I+cells (FIG. 3B; G1) shifted slightly compared with a control sample, which was stained with Thy-I antibody alone, indicating that Thy-I+pup testis cells could be described as av-integrin . Three fractions, Thy-1+av-integrin /dim (G1), Thy-l av-integrin-ldim (G2), and av-integrin+(G3), were isolated by FACS from pup or neonate testis cells, and were transplanted into recipient testes. Donor-derived spermatogenic colonies were generated almost exclusively from Thy-I+(Xv-integrin-/dim cells in both pup and neonate testes (FIG. 3C, FIG. 4C). Pup and neonate Thy-1 +xv-integrin-/dim cells produced 124 and 17 colonies per 105 cells transplanted, respectively. Thy-1+av-integrin-/dim cell fractions did not contain P2M+cells. Therefore, the SSCs is 32M in pup and neonate. In addition, all P2M- Thy-l+a-integrin-/dim cells were a6-integrin+. These results demonstrate that p2M- Thy-I+(x6-integrin+subpopulations contain the majority of SSCs in wild type adult, pup, and neonate testes as well as cryptorchid adult testis. Stem cell enrichment by Thy-I antibody-coniugated magnetic microbeads FACS analysis of cryptorchid adult, wild type adult, pup, and neonate testes showed that Thy-I+cells other than the SSCs enriched subpopulations were very few in the testes (FIGS. 1-4). Therefore, it was determined that SSCs could be enriched by MACS with Thy-I antibody-conjugated magnetic microbeads, which would greatly simplify stem cell enrichment at all ages. Unfractionated testis cells and Thy-I+cells that were isolated by Thy-i microbeads (MACS Thy-I+) from cryptorchid adult, wild type adult, pup, and neonate testis cells were transplanted into recipient testes to determine the stem cell activity of MACS Thy-l+cells. The number of donor-derived spermatogenic colonies in the recipient testes is shown in FIG. 5. MACS Thy-I+cells of cryptorchid adult, wild type adult, pup, and neonate produced 192, 48, 70, and 22 colonies per 105 cells transplanted, respectively. Compared with unfractionated cell populations prior to MACS separation, the stem cells in the MACS Thy-l+fraction were enriched 6-fold, 30-fold, 4-fold, and 5-fold for cryptorchid adult, wild type adult, pup and neonate, respectively. These results indicate that Thy-I microbeads enriched S SCs efficiently and that the highest concentration of stem cells was achieved when cryptorchid adult testes were used. Establishment of a culture system to support SSC expansion After methods were developed to obtain an enriched population of stem cells, experiments were initiated to evaluate in vitro conditions to maintain and/or expand stem cell number ex vivo. A major objective was to develop a culture system that consisted of defined conditions to facilitate studying the biology of the stem cell and identifying external factors for in vitro maintenance. In a previous study with primitive hepatic progenitor cells, a combination of serum-free hormonally defined medium and STO feeders supported clonal growth of purified progenitors, and it was possible to study fate determination of the progenitors in vitro by modifying the culture condition (Kubota et al., Proc. Natl. Acad. Sci. U.S.A., 97:12132-7 (2000)). Therefore, the basic culture system that consisted of enriched stem cells, serum-free defined culture medium, and mitotically inactivated STO feeders was employed in this study. Because large numbers of contaminating non-stem cells are likely to influence SSC behavior in culture, an enriched stem cell population was believed to be critical to evaluate different culture conditions. Therefore, P2M Thy-l+cells isolated by FACS from cryptorchid testes were chosen as a starting SSC population, because the stem cell activity was.highest among the Thy-I+populations examined.

Initially, the effects of FBS, basal medium type, and feeder cells were evaluated on stem cell activity. Four different concentrations of FBS, 0%, 0.1%, 1%, and 10%, were compared. Previous studies had shown that MEMO was better than DMEM for maintenance of SSCs (Nagano et al., Biol. Reprod., 68:2207-2214 (2003)); therefore, two basal media, MEMO and FIO, were examined. Originally, FIO was developed for clonal culture under serum-free condition (Ham, Exp. Cell Res., 29:515-26 (1963)). In addition, it has been reported that only FIO, but not other basal media tested, was able to maintain immature stages of mesenchymal cells (Rando et al., J. Cell Biol., 125:1275-1287 (1994)). STO cells, which are able to maintain various types of stem and progenitor cells (Nagano et al., Tissue Cell, 30:389-97 (1998), Robertson, E. J., Oxford, England: IRS Press, 71-112 (1987), Kubota, H., et al., Proc. Natl. Acad. Sci. U.S.A., 97: 12132-7 (2000)), as well as NB testis cells, were used as feeder cells. NB testis feeder cells may produce critical factors for expansion of SSCs, because the number of SSCs increases dramatically after birth (Shinohara et al., Proc. Natl. Acad. Sci. U.S.A., 98:6186-91 (2001)).

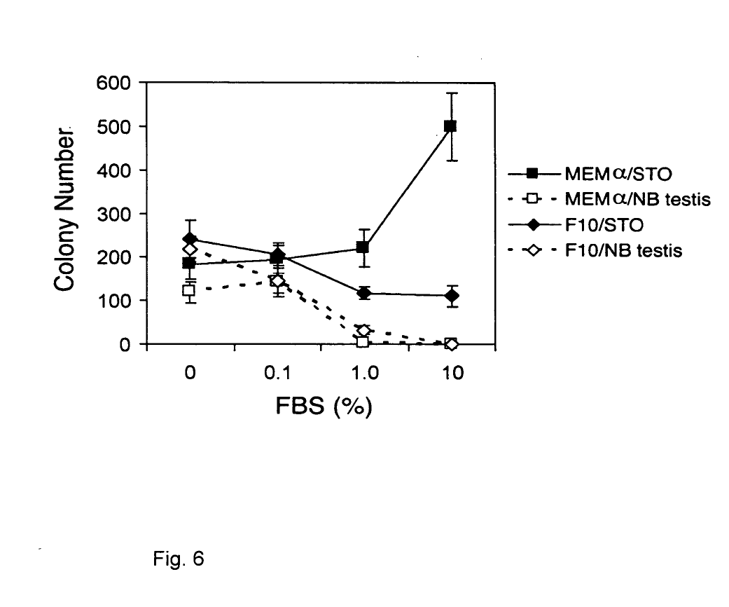

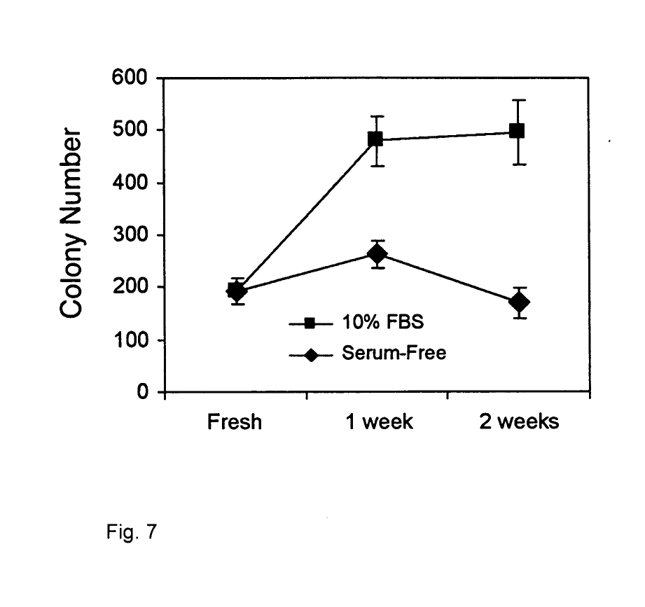

In total, 16 conditions (4 serum concentrations x 2 basal media x 2 types of feeders) were compared. In each condition, 2 x 104, 2M Thy-14+cells, which were expected to generate about 280 spermatogenic colonies per 10⁵ cells transplanted (FIG. 1C), were cultured in two wells of a 6-well plate (1000 cells/cm²) in which feeder cells were seeded. After 8 to 10 days of culture, all cells were harvested from the two wells and transplanted into recipient testes to determine whether SSCs increased or decreased during the culture periods by comparing the number of spermatogenic colonies generated in recipient testes to the 280 value in FIG. 1C. In the absence of FBS, between 42% (MEMx/NB testis) and 85% (FIO/STO) of SSCs were maintained during the 8-10 day culture period (FIG. 6). However, in the presence of serum, the SSC activity markedly decreased in combination with NB testis feeders. In the presence of 10% serum, there were no colonies generated in either the MEMa/NB testis or FIO/NB testis combination. Interestingly, SSCs expanded about 1.8-fold (500/283) in the presence of 10% FBS when MEMo and STO feeders were used, while the number of SSCs in F1O/STO/10% FBS decreased about 0.4-fold (111/283). These results indicated that combinations of different basal medium, FBS concentration, and types of feeder cells had a dramatic effect on self-renewal of SSCs in vitro. Identification of growth factor effect on SSCs in vitro Although FPO maintained SSCs in serum-free medium, SSC expansion was observed only in the MEMo/STO/10% FBS condition. This result suggested that additional soluble factors (i.e., from FBS) could support self-renewal of SSCs in the MEMo/STO serum-free condition. Therefore, the MEMa/STO combination was used for further experiments to develop a serum-free defined culture condition to support expansion of SSCs. Because the protocol for obtaining subpopulations of SSCs is more rapid and flexible using MACS than FACS, the results of the culture experiments with FACS-enriched SSCs (FIG. 6) were confirmed using MACS Thy-1+cells in MEMa/STO/serum-free and 10% FBS culture medium. Freshly isolated MACS Thy-1+cells, one-week cultured cells, and two-week cultured cells were transplanted into recipient testes for subsequent analysis of colony formation. In 10% FBS, a 2.5-fold (478/191) expansion of SSCs was observed following one week culture, but no change occurred from the first to second week (FIG. 7). In the serum-free condition, SSCs expand slightly (1.3-fold; 261/191) in one week although the number of SSCs decreased to 0.7-fold (169/191) in the second week of the culture. These results indicate that maintenance and proliferation of SSCs in the MACS Thy-1+cells was similar to that in FACS-enriched p2M- Thy-1+cells; therefore, MACS was used in subsequent experiments to provide SSC populations.

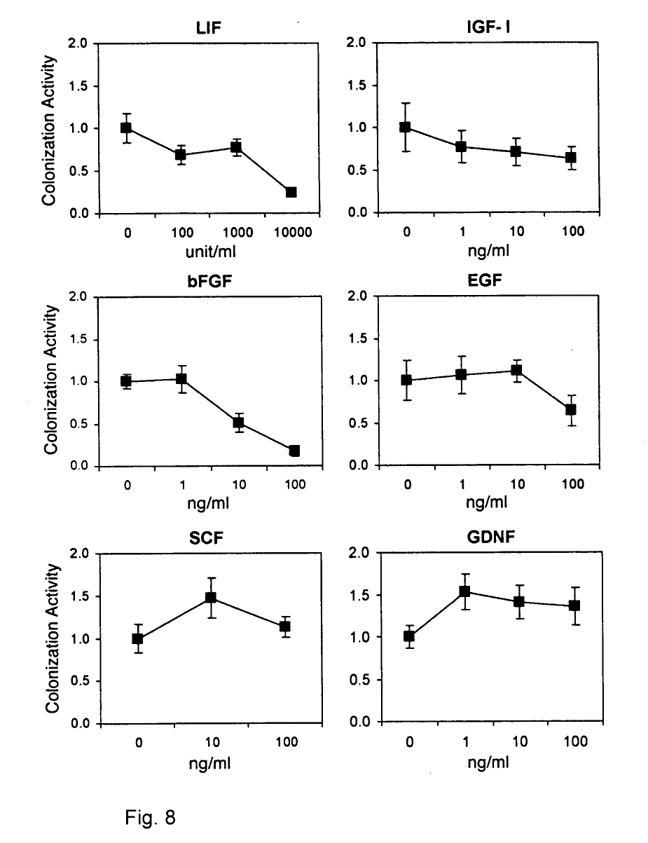

On the basis of the above experiments, in which SSCs were maintained without loss of activity for the first week in the serum-free culture condition, a system was established to study SSC maintenance and proliferation in vitro. This system consisted of STO cell feeders and MEMo, and the SSC enriched population was Thy-1+cells from MACS. Using this system, the effect of 6 growth factors at 2 to 3 concentrations on SSC proliferation during a 7-day period was examined in vitro (FIG. 8). Two factors, LIF and bFGF, did not have a significant effect on SSC activity during the 7- day period at the lowest concentrations. However, stem cell activity was significantly decreased in 1000 units/ml (p<0.009) LIF and in lOng/ml (p<0.01) or 100 ng/ml (p<0.0003) bFGF. The other four factors (EGF, IGF-l, SCF, and GDNF) did not demonstrate a significant effect on SSC activity during the culture period at the concentrations examined, although both GDNF and SCF increased stem cell activity at each of concentrations employed. Therefore, this assay system, using defined serum-free culture conditions, provides a precise assessment of the direct effect of individual growth factors, and the approach can be readily extrapolated to multifactor analyses.

These results demonstrate that the above-described assay system comprising defined serum-free culture conditions provides a precise assessment of specific biological actions of individual growth factors on SSCs, and the approach can be readily extrapolated to multifactor analyses.

One feature of the present invention is the establishment a model culture system for studying regulatory mechanisms of self-renewal and differentiation of SSCs in vitro. For this purpose, we minimized unknown characteristics in the culture system. First, enriched cell populations of SSCs were selected to eliminate the effect of testicular somatic cells or differentiating germ cells. Second, a serum-free defined medium was used. Third, mitotically inactivated STO cells, which are a well-established cell line, were chosen for feeders instead of heterogeneous embryonic fibroblasts or testis cells. In the end, a testis cell population with a high concentration (5-10%) of SSCs was identified, and a culture condition that consisted of serum-free medium and STO feeders was established. Under these culture conditions, SSCs were maintained in vitro without significant loss of stem cell activity for at least one week.

One feature of the present invention was the definition of characteristics of the stem cell that would facilitate its identification and purification. Such characteristics comprise one or more surface antigens and that the antigens might be conserved throughout life as has been found for hematopoietic stem cells (Ikuta et al., Cell, 62:863-874 (1990)). It was previously demonstrated that SSGs in cryptorchid testes were MHG-I Thy-1+, almost all the stem cells were contained in this surface antigenic profile, and about 1 in 15 cells of this population behaved as a fully functional stem cell when transplanted to recipient testes (Kubota et al., PNAS., 100:6487-6492 (2003)). However, cryptorchid testes are an enriched source of SSCs, and it could be argued that these testes represent a special or unusual physiological environment. Therefore, experiments set forth herein used FACS analysis of testis cells and a transplantation assay to demonstrate that stem cell activity is found almost exclusively in 02M- Thy-1+cells of neonatal, pup, and adult testes. Because 02M is a light chain of MHC-1, the surface phenotype of SSCs is MHC-1- Thy-I+throughout postnatal life of the mouse. Moreover, MHC-1- Thy-1+cells of neonate, pup and adult testis share the surface phenotype of (x6-integrin+and av-integrin- (Kubota et al., PNAS., 100:6487-6492 (2003)). Although the surface phenotype of other testis cells varies during development, it was possible to use specific purification techniques to prove that the SSCs have a distinctive surface phenotype that allows enrichment for culture and other studies. Furthermore, the continuity of surface phenotype suggests that other unique biochemical molecules of SSCs can be identified beginning with these MHC-1- Thy-1+a6-integrin+xv-integrin-/di^{m} cells.

Stem cell activity in the MHC-1-Thy-1+cell population changed during development; it was low (17 colonies/10⁵ cells transplanted) in the neonate but increased dramatically (124 colonies/10⁵ cells transplanted) in the pup. The only germ cells in the neonate testis are gonocytes, located in the center of the seminiferous tubule (Nagano et al., APMIS, 106:47-55 (1998), Vergouwen et al., J. Reprod. Fertil., 99:479-85 (1993)). By 6 dpp, most of the gonocytes have migrated to the basement membrane and become stem cells or type A spermatogonia (de Rooij, Int. J. Exp. Pathol., 79:67-80 (1998), Nagano et al., Anat. Rec., 258:210-220 (2000)). In this invention, 0.5-1.5 dpp neonates and 4.5-5.5 dpp pups were used. Testis size increases little during this period (-5.1 x 10⁵ cells/testis at 0.5-i .5 dpp and -9.6x10⁵ cells/testis at 4.5-5.5 dpp); therefore, the dramatic increase of stem cell activity in the MHC-1- Thy-1+ov-integrin-/dim cells represents about 4 doublings in 4 to 5 days. A similar doubling rate can be obtained from values for unfractionated cells in FIG. 5 (Pup=14.4 vs Neonate=1.7). Therefore, the stem cells are dividing approximately everyday, which is about 4 times or more rapid than the 4 to 5 day doubling time estimated in adult testis (de Rooij, Int. J. Exp. Pathol., 79:67-80 (1998), van Keulen et al., Cell Tissue Kinet., 8:543-551 (1975)). Subsequently, little change in stem cell concentration occurs in the cell population from pup to wild type adult. Such a dynamic alteration of stem cell activity in this phenotypically identical cell population provides a unique opportunity to investigate SSG development.

The stem cell activity of MHC-1-Thy-1+x6-integrin+cells of wild type adult testis (162 colonies/10⁵ cells transplanted) was only slightly higher than in the 5-day pup, which indicates that the relationship between stem cell and number of associated primitive spermatogonia with similar surface phenotype is established soon after birth. Additionally, MHC-l-Thy-1+cells of the cryptorchid testis contained a much higher stem cell activity (283 colonies/10⁵ cells transplanted). The difference in the microenvironment of the cryptorchid and wild type testis must result in a different ratio of stem cell to primitive spermatogonia with MHC-I-Thy-I+surface phenotype. A negative feedback system to inhibit the proliferation of undifferentiated spermatogonia by differentiating spermatogonia has been reported in wild type testis (de Rooij et al., Cell Tissue Kinet., 18:71-81 (1985). However, experimental cryptorchid testes are devoid of all differentiated spermatogonia, only undifferentiated spermatogonia remain in the testis (Nishimune et al., Fertil. Steril., 29:95-102 (1978)). Thus, there is no such feedback system present in the cryptorchid testis, and under these conditions turnover of undifferentiated spermatogonia may be more active (Tadokoro et al., Mech. Dev., 113:29-39 (2000)). Moreover, the loss of differentiating germ cells profoundly influences the balance between the germ cell and endocrine compartments of the testis, and absence of differentiating germ cells interferes with the reestablishment of spermatogenesis in irradiated rat testes even when stem cells remain (Kangasniemi et al., Biol. Reprod., 54:1200-1208 (1996)). These physiological differences between cryptorchid and wild type adult testes must influence the ratio of stem cells to non-stem MHC-1-Thy-l+cells, and result in the difference we observed in stem cell activity. Although stem cell activity and the microenvironment surrounding SSCs change dramatically after birth and are quite different in adult wild type and cryptorchid testis, Thy-i is expressed on SSCs constitutively in each of these conditions. Moreover, Thy-l has been identified as a marker on rat SSCs, and may represent a characteristic surface phenotype of SSCs for all species. The biological function of Thy-i on SSCs and primitive germ cells is unknown, and studies on hematopoietic and neuronal systems have also failed to elucidate the exact role of this surface antigen.

Following identification of the surface antigens for SSCs by FACS, the phenotype was exploited to obtain enriched populations of SSGs using microbeads. This later technique is simple, less expensive, and can produce more cells in less time, which facilitates assessment of various culture conditions. Although there are several known antigenic markers of SSCs including a6-integrin, CD24, or D 1 -integrin (Kubota et al., PNAS., 100:6487-6492 (2003), Shinohara et al., Proc. Natl. Acad. Sci. U.S.A., 97:8346-51 (2000), Shinohara et al., Proc. Natl. Acad. Sci. U.S.A., 96:5504-9 (1999)), Thy-I expression is relatively unique for the SSC population. For example, in the pup testis, a number of cc6-integrin+av-integrin+or CD24+oxv-integrin+cells, which are not SSCs, were identified. However, selective expression of Thy-I on SSCs in the testis allowed the use of Thy-I microbeads for enrichment of SSCs by MACS. In fact, significant enrichment of SSCs from cryptorchid adult, wild type adult, pup, and neonate testis was achieved (FIG. 5). In both FACS and MACS systems, the Thy-I+fraction of cryptorchid adult testes contained the highest stem cell activity. Therefore, SSC populations of cryptorchid testes enriched by FACS and MACS were used for all culture experiments, which minimized the effect of non-stem cells on the culture environment.

While it is desirable to minimize the effect of non-stem cells of the testis on culture conditions, neonatal testis cells are different than those from adult wild type or cryptorchid males. The rapid increase in stem cell concentration in the MHC-I Thy-14+av-integrin-dim cell population during the first 5 days following birth suggests that testis somatic cells are producing growth factors to stimulate the stem cell population. Therefore, a comparison of NB testis to STO feeders on SSC maintenance in vitro was undertaken. When SSCs enriched by FACS were cultured with NB testis feeders in the presence of serum at a concentration of 1 or 10%, the number of stem cells decreased dramatically during 8-10 days culture, and no stem cell activity was detected in 10% FBS using NB. testis feeders (FIG. 6). This result suggests that stimulation of NB testis feeders by FBS resulted in production of factors that induce differentiation or cell death in SSCs, an effect not anticipated from the increase in stem cell activity found in pup. These results further suggest that reduction of the testicular somatic cell population improves culture conditions for ex vivo expansion of SSCs in the presence of serum. However, a combination of serum-free medium and NB testis feeder cells did not show a detrimental effect on SSC maintenance, suggesting that serum-free conditions dramatically alter the NB testis cell effect on SSCs.

The combination of serum with NB testis feeder was detrimental to SSC maintenance in vitro, and serum in combination with FIO and STO feeders also appeared to have a negative effect. However, serum added to MEMa with STO feeders enhanced the maintenance and proliferation of SSCs in vitro. These results indicate that serum is a complicating factor in developing culture conditions for SSCs and in understanding the environmental and growth factor requirement for SSC proliferation. Nonetheless, the very positive effect of 10% FBS in MEMA using STO feeders suggested that FBS contained factors beneficial for SSC proliferation and that MEMA and STO feeders could serve as a culture condition in which to test the effect of individual growth factors when added to supplemented serum-free MEMA.

With the use of MEMA serum-free medium and STO feeders, the stem cell number did not decrease appreciably during the 7 day culture period (FIG. 7). Therefore, the culture system allowed examination of the effect of individual growth factors on replication of SSCs in vitro. Six growth factors (IGF-I, LIF, bFGF, EGF, SCF, and GDNF) were chosen to evaluate the system for studying SSC proliferation and biology in vitro. Two growth factors, IGF-I and LIF, appeared to have a more negative than positive effect on SSC maintenance in vitro at concentrations often employed for other cells in culture. Particularly surprising was the absence of a supporting role for LIF, which can replace the STO feeder effect when maintaining ES cells in vitro (Smith et al., Nature, 336:688-690 (1988), Williams et al., Nature, 336:684-687 (1988)). The influence of EGF and bFGF is more equivocal. EGF appears neutral up to a level of 1 to 10 ng/ml but may be inhibitory at 100 ng/ml, while bFGF is inhibitory at levels (10 ng/ml) used to support primordial germ cells in vitro (Matsui et al., Cell, 70:841-847 (1992)). Another study, however, suggested that a peak response of bFGF for PGC proliferation is at around 1 ng/ml (Resnic et al., Nature, 359:550-1 (1992)). The two growth factors that appeared to have a beneficial effect on SSC maintenance were SCF and GDNF. Addition of SCF appeared to increase the number of SSCs even though there is no detectable expression of the receptor, c-kit, on the cell surface of SSCs by FACS analysis (Kubota et al., PNAS., 100:6487-6492 (2003)). It cannot be ruled out that a small number of receptors may be present on the cell surface, and further study for a longer time period and in combination with other factors will be useful. In the presence of all concentrations of GDNF, the number of stem cells was increased approximately 1.5-fold, indicating that proliferation of SSCs may have occurred during the one week culture period. In vivo studies with transgenic animals (Meng et al., Science, 287:1489-1493 (2000)) or transfection by electroporation (Yomogida et al., Biol. Reprod., 69:1303-1307 (2003)) strongly suggest that GDNF is involved in SSC proliferation. Furthermore, previous studies using GDNF at 100 ng/ml in serum containing medium also showed a significant increase in SSC activity after one week. Likewise, GDNF forms part of a growth factor cocktail added to the serum-supplemented condition that supports SSC proliferation from neonatal ICR or C57/BL/6 x DBA/2 mouse testes (Kanatsu-Shinohara et al., Biol. Reprod., 69:612-616 (2003)), although LIF, EGF, and bFGF, which are the remaining growth factors in the cocktail, appeared to have no positive effect in the culture studies described here. The effect of GDNF on SSCs in our previous study in which serum was used and in this study in a well defined serum-free medium strongly suggests that GDNF should be a component of the basal medium for SSCs. Experimental Example 2: Growth Factors Required for Self-Renewal and Expansion of Mouse Spermatogonial Stem Cells (SSCs) Donor Mice and Cell Collection.

Two transgenic mouse lines expressing reporter genes, B6.129S7-Gtrosa26 (designated ROSA; The Jackson Laboratory; Bar Harbor, ME) and C57BL/6-TgN(ACTbEGFP)1 Osb (designated C57GFP; The Jackson Laboratory; Bar Harbor, ME) were used to distinguish donor cells from recipient cells after transplantation. ROSA mice express the Escherichia coli lacZ gene that encodes a gal protein in virtually all cell types including all stages of spermatogenesis (Tegelenbosch et al., Mutat. Res., 290:193 (1993)). Donor ROSA cells are identified by staining with the P-gal substrate, 5-bromo-4-choloro-3-indolyl P-D-galactoside (X-gal). C57GFP mice express a GFP reporter gene under the control of the chicken ,-actin promoter and cytomegalovirus immediate early enhancer (Brinster et al., Proc. Natl. Acad. Sci. U.S.A., 91:11303 (1994)). C57GFP is expressed in most cells of this mouse (Brinster et al., Proc. Natl. Acad. Sci. U.S.A., 91:11303 (1994)). Wild-type mouse lines used were DBA/2J, C57BL/6, SJL, and 129/SvCP (all from The Jackson Laboratory; Bar Harbor, ME). Pup testis cells (5-8 days postpartum, dpp; day of birth is 0.5 dpp) were collected from the hemizygous transgenic mice, DBA/2J x ROSA, C57BL/6 x ROSA, or C57GFP x ROSA and from inbred wild-type mice, including C57BL/6, SJL, and 129/SvCP. Cell suspensions from pup testes were prepared by enzymatic digestion (Kubota et al., Biol. Reprod. 10.1095/biolreprod.104.029207 (2004)). To enrich SSCs, magnetic-activated cell sorting (MACS) was employed as describe previously (Kubota et al., Proc. Natl. Acad. Sci. U.S.A., 100:6487 (2003)). Briefly, pup testis cells were fractionated by Percoll centrifugation, and the cells in the pellet were incubated with magnetic microbeads conjugated to anti-Thy-1 antibody (Miltenyi Biotec) (Kubota et al., Proc. Natl. Acad. Sci. U.S.A., 100:6487 (2003)). Thy-l+cells were selected by an MS separation column (Miltenyi Biotec) according to the manufacture's protocol. Cell Culture The culture system consisted of serum-free medium and mitotically inactivated STO cell feeders as described (Meng et al., Science, 287:1489 (2000)) and modified for SSCs (Kubota et al., Proc. Natl. Acad. Sci. U.S.A., 100:6487 (2003)). The serum-free medium for SSCs consisted of minimum essential medium-alpha (Invitrogen; Carlsbad, Calif.) to which was added 0.2% bovine serum albumin (MP Biomedicals; Irvine, Calif.), 5 pg/ml insulin, 10 μg/ml iron-saturated transferrin, 7.6 peq/l free fatty acids mixture (Yomogida et al., Biol. Reprod., 69:1303 (2003)), 3×0-8 M H₂SeO₃, 50 μM 2-mercaptoethanol, 10 mM HEPES, 60 μM Putrescine (all from Sigma, St. Louis, MO), 2 mM glutamine, 50 units/ml penicillin and 50 1g/ml streptomycin (all from Invitrogen; Carlsbad, Calif.) as described previously (Kubota et al., Proc. Natl. Acad. Sci. U.S.A., 100:6487 (2003)). Thy-I microbead-selected cells by MACS were cultured on STO feeders in wells of a 12-well plate at densities of 6-10 x 104 cells/well in 1.5 ml of the serum-free medium with or without growth factors as indicated. Human GDNF. (R&D systems), rat GFRA-Fc fusion protein (GFRAl-Fc, R&D systems; Minneapolis, MN), and human bFGF (BD Biosciences; San Jose, Calif.) were used at a final concentration of 40 ng/ml, 300 ng/ml, and 1 ng/ml, respectively. Cells were subcultured at 5-7 day intervals at a 1:2-4 dilution. For experiments to examine the effect of soluble factors on proliferating SSCs, mouse LIP (Chemicon International; Temecula, Calif.), mouse SCF (R&D systems; Minneapolis, MN), mouse EGF (BD Biosciences; San Jose, Calif.), human IGF-I(R&D systems; Minneapolis, MN), and mouse Noggin-Fc fusion protein (R&D systems; Minneapolis, MN) were used at a final concentration of 103 units/ml, 60 ng/ml, 10 ng/ml, 10 ng/ml, and 300 ng/ml, respectively. For culture with PBS, serum supplemented medium was prepared by adding heat inactivated (56° C., 30 mm) FBS (Hyclone; Logan, UT) to the serum-free medium. All cultures were maintained at 37° C. in a humidified 5 % C0₂ atmosphere. The medium was changed every 2-3 days. Fluorescence Activating Cell Sorting Cell staining for flow cytometry of freshly isolated testis cells or cultured cells was performed as described previously (Kubota et al., Proc. Natl. Acad. Sci. U.S.A., 100:6487 (2003), Jeong et al., J. Androl., 24:661 (2003)). Antibodies used for surface antigens were; biotin-conjugated anti-Thy-i (53.2.1, BD Biosciences; San Jose, Calif.), allophycocyanin (APC)-conjugated anti-a6-integrin (GoH3, BD Biosciences; San Jose, Calif.), R-phycoerythrin (PE)-conjugated anti-av-integrin (RMV-7, H9.2B8, BD Biosciences; San Jose, Calif.), biotin-conjugate anti-c-Kit (2B8, BD Biosciences; San Jose, Calif.), anti-gpl30 (RM PI, MBL International), anti-NCAM (H28-123-16, Cedarlane), and anti- GFRa1 (81401.11, R&D systems; Minneapolis, MN) antibodies. Alexa Fluor 488-conjugated streptavidin, Alexa Fluor 647-conjugated goat anti-mouse IgG2b antibody, and Alexa Fluor 647-conjugated goat anti-rat IgG antibody (all from Molecular Probes; Eugene, OR) were used as secondary reagents. Stained cells were analyzed by FACS Calibur (BD Biosciences; San Jose, Calif.). Immunohistochemistry and Alkaline Phospatase Activity Assay Cultured germ cells were fixed with 4% paraformaldehyde for 20 mm and permeabilized with 0.1% Triton X-100 in PBS for 60 mm for immunohistochemistry. For GCNA1 expression, fixed and permeabilized cultures were stained with rat anti-GCNA1 antibody (IOD9G1 1), followed by staining with secondary alkaline phosphatase-conjugated goat anti-rat 1 gM antibodies (Pierce; Rockford, IL). Color development was performed with alkaline phosphatase enzyme substrate (nitro blue tetrazolium and 5-bromo-4-chloro-3 -indolyl phosphatase; Promega; Madison, WI). The staining procedure was confirmed not to detect endogenous AP activity. For c-Ret expression, mouse anti-human c-Ret monoclonal antibody (132507, R&D systems; Minneapolis, MN) and goat anti-human c-Ret polyclonal antibody raised against the tyrosine kinase domain (sc-167-G, Santa Cruz Biotechnology; Santa Cruz, Calif.) were used. Following primary antibody-staining, cells were stained with Alexa Fluor 488-conjugated goat anti-mouse IgG, antibody for anti-c-Ret monoclonal antibody or Alexa Fluor 488-conjugated donkey anti-goat IgG antibody for anti-c-Ret polyclonal antibody. For Oct-4 expression, cultured germ cells and ES cells (AB 1; The Wellcome Trust Sanger Institute, UK) were fixed as above and stained with goat anti-human Oct-4 polyclonal antibody (R&D systems; Minneapolis, MN), followed by staining with Alexa Fluor 488-conjugated donkey anti-goat IgG antibody. For detection of endogenous AP activity in cultured germ cells and ES cells, cultures were fixed with 66% acetone and stained with naphthol AS-BI phostate/fast red violet solution (Chemicon International; Temecula, Calif.). Testis Cell Transplantation and Analysis of Recipient Testes. Donor testis cells were resuspended in serum-free medium and transplanted into the testes of NCr nude male mice (nu/nu, Taconic) or immunologically compatible C57BL/6 x 129/SvCP Fl hybrid recipient male mice (Kanatsu-Shinohara et al., Biol. Reprod., 69:612 (2003), Nagano et al., Biol. Reprod., 68:2207 (2003)). Nude and C57BL/6 x 129/SvCP Fl recipient mice were treated with busulfan (44 mg/kg and 55 mg/kg, respectively) 4-6 weeks before use (Kanatsu-Shinohara et al., Biol. Reprod., 69:612 (2003), Nagano et al., Biol. Reprod., 68:2207 (2003)). Approximately 10 p.1 of donor cell suspension was introduced into the. seminiferous tubules of each recipient testis, resulting in 70-80% filling of the tubules (Kubota et al., Biol. Reprod. (2004). (available on line at <<http://www.bio 1 reprod.org/cgi/rapidpdf/biolreprod.104.029207vl >>)). Recipient testes were collected 2 months after donor cell transplantation and analyzed by X-gal staining (Materials and methods are available as supporting material on Science Online)). The number of donor-derived spermatogenic colonies was counted using a dissection microscope. To restore fertility of infertile animals, approximately 2 μl of the donor cell suspension (−30×10⁶ cells/ml) were injected into W⁵⁴ /W mouse pup testes (Endersand et al., Dev. Biol., 163:331 (1994)). Experimental procedures were conducted in accordance with the Guide for Care and Use of Laboratory Animals from the National Academy of Science.

A mixed effects ANOVA model with fixed effects for treatment group, testis side, and experiment number, and a random effect for mouse, was used to compare the mean colonies per 105 cells. Bonferroni adjusted p-values were used to determine significant differences for each of the pairwise comparisons versus the control group (Kubota et al., Proc. Natl. Acad. Sci. U.S.A., 100:6487 (2003)).

Prior to culture, MACS Thy-i cells obtained from C57xROSA pup testes were stained with antibodies for a6-integrin, xv-integrin, and Thy-I to compare with the phenotype of proliferating SSCs in culture. FACS analysis indicated that approximately 70% of cells were av-integrinn , and these cells were a6-integrin+Thy-1 Io+(FIG. 12). Essentially all stem cell activity in pup testes is present in this cell population (Kubota et al., Proc. Natl. Acad. Sci. U.S.A., 100:6487 (2003)). The fresh MACS Thy-1 cells were transplanted into recipient testes and produced 342 33 (mean±SEM, n =6) colonies' per 10⁵ cells; therefore the colony number per 10 av-integrinn/d Q6-integrin+Thy-1 Io+cells is 478±46 (mean +SEM, n =6, 342/0.716; Table 1).

TABLE 1
Stem cell activity and phenotypic analysis of cultured spermatogonial stem cells

| | | | | Colony number per 10⁵ av- |
|---|---|---|---|---|
| | | | Proportion of αv- | integrin^{-/dim} |
| | Number of | Colony number per | integrin^{-/dim} α6- | α6-integrin⁺ |
| Days in | testis | 10⁵ cells | integrin⁺ Thy-1^{lo/+} cells | Thy-1^{lo/+} cells |
| culture | analyzed | transplanted^{†} | (%)‡ | transplanted^{§} |
| 0 | 6 | 342 ± 33 | 71.6 | 478 ± 46 |
| 15 | 6 | 102 ± 11 | 25.4 | 400 ± 44 |
| 28 | 6 | 105 ± 12 | 19.7 | 532 ± 63 |
| 42 | 6 | 75 ± 18 | 12.1 | 616 ± 146 |
| 56 | 6 | 71 ± 14 | 18.8 | 377 ± 76 |
| 92 | 6 | 207 ± 35 | 40.1 | 516 ± 86 |

^{†}The number of spermatogenic colonies generated by 10⁵ cells transplanted into recipient testes (mean ± SEM).
‡Proportion of αv-integrin^{-/dim} α6-integrin⁺ Thy-1^{lo/+} cells in cell suspension transplanted was determined by FACS analysis.
^{§}The number of spermatogenic colonies generated by 10⁵ αv-integrin−/^{dlm} α6-integrin⁺ Thy-1^{lo/+} cells transplanted into recipient testes (mean ± SEM).

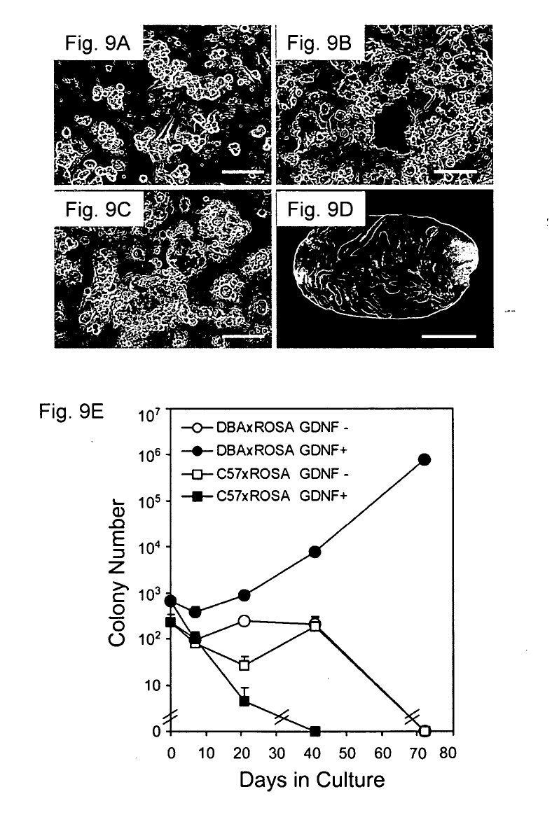

The MACS Thy-i cells were placed on STO feeders in serum-free medium containing GDNF, soluble GFRα1, and bFGF. After 2-weeks culture, cells were harvested and stained with antibodies for a6-integrin, αv-integrin, and Thy-I to determine the surface phenotype of germ cells growing in clumps by FACS analysis. STO cells express av-integrin; moreover, mitotically inactive STO feeders are large with considerable cytoplasmic structure. Therefore, STO feeder cells could be identified readily as side scatter^{hi} αv-integrin+cells and were gated out (FIG. 12). The remaining population, clump-forming germ cells, was side scatter^{hi} αv-integrin/di, and the surface phenotype was (x6-integrin+Thy- 10+(FIG. 12). The expression pattern of cc6-integrin and Thy-I in the xv-integrinn/dim cells was similar to that of fresh MACS Thy-I cells (FIG. 12). The cultured cell population was transplanted to determine stem cell activity, and they produced 102±11 (mean +SEM, n =6) colonies per 10⁵ cells transplanted (Table 1). FACS analysis of these cells indicated that 25.4% of the cell population was av-integrin-/dim a6-integrin+Thy-o¹/⁺therefore, 102 colonies per 105 cells transplanted represents 400 colonies per 105 cells (Table 1). A fraction of the original cell population was maintained in culture and analyzed at 4, 6, 8, and 13 weeks (Table 1). The antigenic phenotype of clump-forming cells did not change during the culture period. Because of STO feeder cell content, the percentage of αv-integrin-/dim 6-integrin+Thy-10/+cells in culture was different at each time point, and colony numbers per 10⁵ cells transplanted ranged from 71 to 207 during the 13-week culture period. However, stem cell activity calculated by colony number per 105 av-integrin/dim a6-integrin+Thy-10/+cells was relatively constant (488+41, mean±SEM, n =30, day 15 to 92) and similar to the value for freshly isolated MACS Thy-1 cells (478±46, mean±SEM, n =6, Table 1). Factors responsible for cell fate determination of SSCs SSCs were enriched by magnetic activated cell sorting with anti-Thy-1 antibody (MACS Thy-I cells) from pup testes (Kubota et al., Biol. Reprod. 10.1095/biolreprod.104.029207 (2004)), which were obtained by mating β-galactosidase (β-gal) expressing mice (ROSA) to DBA/2J or C57BL/6 mice (DBAxROSA or C57xROSA, respectively). Enriched SSCs isolated from pups of each strain were placed onto STO feeders in a serum-free defined medium (Kubota et al., Biol. Reprod. 10.1095/biolreprod.104.029207 (2004)) with or without GDNF (40 ng/ml) (Materials and methods are available as supporting material on Science Online)). In the presence of GDNF, some MACS Thy-I cells divided and formed clumps with tight intercellular contacts resulting in a ball or mass of cells. Every 5-7 days, the clumps were dissociated with trypsin and subcultured. DBAxROSA pup MACS Thy-i cells continued dividing and regenerated clumps in the subculture (FIG. 9A); whereas, C57xROSA MACS Thy-i cells formed small clumps after the first subculture, which gradually disappeared with subsequent subculturing. DBAxROSA pup cells without GDNF also gradually disappeared. The clump-forming cells were stained with an antibody against germ cell nuclear antigen (GCNA 1) (FIG. 9B), which is a germ cell specific marker (Endersand et al., Dev. Biol., 163:331 (1994))), indicating that the clumps consisted of germ cells. When cultured cells were incubated with X-gal, expression of β-gal was seen specifically in clumps (FIG. 9C). A continuous increase in cell clump number was observed, suggesting that they contained SSCs. Therefore, after 1-, 3-, 6-, and 10-weeks in vitro, cultured cells were transplanted into the seminiferous tubules of busulfan-treated nude mouse testes to determine the ability of the expanding germ cells to form colonies of spermatogenesis. Only SSCs are able to generate colonies of complete spermatogenesis (Brinster et al., Proc. Natl. Acad. Sci. U.S.A., 91:11303 (1994))). Two months after transplantation, donor-derived colonies of spermatogenesis in recipient testes were counted (FIG. 9D). The number of donor-derived spermatogenic colonies produced from each experimental group at the individual time points per 105 cells of MACS Thy-I cells originally placed in culture (day 0), is shown in FIG. 9E. Only germ cell clumps derived from DBAxROSA pups in culture with GDNF continued to generate spermatogenic colonies during the culture period (FIG. 9E). β-gal expressing cells were found only in clumps (FIG. 9C); therefore, the clumps from DBAxROSA pups indeed contained SSCs. After 10-weeks of culture, 105 MACS Thy-1 cells collected from DBAxROSA pups generated 7.6+1.4×10⁵ (mean±SEM, n =6, FIG. 9E) colonies in the presence of GDNF, but no colonization was observed in cultures without addition of the growth factor (FIG. 9E). Cells from C57xROSA pups had no stem cell activity after 10-weeks of culture with or without GDNF (FIG. 9E). These results demonstrate that SSCs of DBAxROSA pups are able to proliferate in serum-free medium and that an essential growth factor is GDNF. However, GDNF alone did not support in vitro expansion of SSCs derived from C57xROSA mice.

Subsequently, additional factors were found to support stem cells of C57xROSA pups. Cellular responses to GDNF are mediated by a multicomponent receptor complex consisting of c-Ret receptor tyrosine kinase and a glycosyl phosphatidylinositol-anchored ligand-binding subunit, GFRI, in many cell types (Airaksinen et al., Nat. Rev. Neurosci., 3:383 (2002)). Although it has been shown that mouse spermatogonia express GFRcxl (Meng et al., Science, 287:1489 (2000))), adding soluble GFRαI molecules might potentiate the stimulatory signal through the c-Ret receptor or modulate the signaling pathways (Paratcha et al., Neuron, 29:171 (2001)). In addition, basic fibroblast growth factor (bFGF), a critical growth factor for primordial germ cells (PGCs) in vitro (Matsui et al., Cell, 70:841 (1992), Resnick et al., Nature, 359:550 (1992), Shamblott et al., Proc. Natl. Acad. Sci. U. S. A., 95:13726 (1998)), was examined. MACS Thy-i cells from C57xROSA pups were cultured with GDNF alone or in the combination with soluble GFRαL (GFRαl-Fc, 300 ng/ml), bFGF (1 ng/ml), or both. The cultured cells were transplanted to recipient mice at specific time intervals, as described above. Again GDNF alone did not support expansion of SSCs from C57xROSA; however, addition of soluble GFRαL along with GDNF improved the maintenance of germ cell clumps and showed modest expansion of SSCs in culture (FIG. 10A). A similar but better response was seen when bFGF was added with GDNF, and the degree of SSC proliferation was clearly greater than that in the combination of GDNF and soluble GFRαl. When all three factors, GDNF, soluble GFRal and bFGF, were added to the culture medium, expansion of the germ cell clumps was dramatically enhanced. Transplantation of the cultures at each time point confirmed that the observed increase in germ cell clumps reflected an increase in SSCs (FIG. 10A).

After 10-weeks of culture with the three factors, 105 MACS Thy-I cells generated 1.04±0.23×10⁶ (mean±SEM, n =6, FIG. 1OA) colonies (similar to the value for DBAxROSA SSCs, see above). Before culture, 105 MACS Thy-i cells from C57B/J6 background mice generated 205+26 (mean +SEM, n =6, FIG. 1OA) colonies. Therefore, the stem cell activity increased 5073-fold (1.04 x 106/205); a single stem cell produced over 5000 copies in 72 days. In this experiment the number of SSCs in culture doubled every 5.8 days (72/log25073). Results from three separate experiments indicated an average doubling rate of 5.6+0.2 days (mean +SEM, n =3). This rate of doubling is similar to that estimated for adult SSCs after transplantation into busulfan-treated testes (Nagano, Biol. Reprod., 69:701 (2003)), suggesting that factor-dependent proliferation of SSCs in vitro closely resembles the process of stem cell replication in vivo following transplantation. Three factors, GDNF, soluble GFRal and bFGF, supported expansion of germ cell clumps from all mouse strains examined, including three inbred strains, 129/SvCP (FIG. 1OB-E), C57BL/6, and SJL, and germ cell clumps could be generated from neonates (1 day postpartum; dpp) and pups (5-8 dpp), as well as cryptorchid and wild type adult mice.

The surface antigenic phenotype of SSCs in the mouse testis is av-integrin-/dim a6-integrin+Thy-110+(Kubota et al., Biol. Reprod. (2004). (available on line at http://www.biolreprod.org/cgi/rapidpdf/biolreprod.104.029207vl), (Kubota et al., Proc. Natl. Acad. Sci. U.S.A., 100:6487 (2003)). Although the antigenic phenotype of SSCs is consistent throughout postnatal life, stem cell activity in the av-integrin-/dim a6-integrin+Thy-11030 population changes depending upon age or physiological condition (Kubota et al., Biol. Reprod. (2004). (available on line at http://www.bio 1 reprod.org/cgi/rapidpdf/biolreprod.104.029207v 1)). The surface phenotype of continuously cultured germ cells was investigated and investigated the stem cell activity was investigated by phenotype. Results of transplantation assays in conjunction with FACS analysis demonstrated that the culture conditions exclusively supported proliferation of av-integrim /dim a6-integrin+Thy- 1I+germ cells without altering the stem cell activity.

Because in vitro expansion of SSCs and formation of germ cell clumps were dependent on GDNF, molecules important in signal transduction of this growth factor were analyzed. Immunohistochemistry for c-Ret receptor tyrosine kinase demonstrated that all cells in germ cell clumps expressed the c-Ret receptor (FIG. 1 lA). In addition, FACS analysis detected expression of GFRali and NCAM, which has been identified recently as an alternative GDNF receptor (Paratcha et al., Cell, 113:867 (2003)), by clump-forming germ cells (FIG. 11B). Thus, at least two possible receptor pathways exist for the GDNF effect on stem cells.

Evolution of SSCs in vivo involves the progression of embryonic stem (ES) cells to PGCs, which give rise to SSCs, and all three cell types contribute to the germline; therefore, they are closely related stem cells. It is well established that ES cells and PGCs express a high level of alkaline phosphatase (AP) and Oct-4, a member of the POU transcription factors (Cooke et al., Methods Enzymol., 225:37 (1993), Pesce et al., Stem Cells, 19:271 (2001)). Following induction of differentiation, the expression of both these molecules is reduced and subsequently lost. In addition, expression of Oct-4 is critical for self-renewal and pluripotency of ES cells (Niwa et al., Nat. Genet., 24:372 (2000)). It was demonstrated that cultured SSCs, particularly small germ cell clumps, had clearly lower AP activity than ES cells (FIG. 11C), but the expression of Oct-4 in cultured SSCs was high and similar to that of ES cells (FIG. 11D). Thus, expression of Oct-4 is likely critical to maintenance of SSCs self-renewal capability and can serve as a marker of SSCs in vitro.

ES cells and PGCs are generally cultured with relatively high concentrations of fetal bovine serum (FBS) (Matsui et al., Cell, 70:841 (1992), Resnick et al., Nature, 359:550 (1992), Shamblott et al., Proc. Natl. Acad. Sci. U.S.A., 95:13726 (1998)); therefore, the effect of FBS on SSCs in vitro was investigated. C57xROSA-derived SSCs that had been maintained with GDNF, soluble GFRal and bFGF were then cultured in the same media to which was added PBS at concentrations of 0, 0.1, 1, and 10 percent for 2 weeks. All concentrations of serum inhibited germ cell clump development, and the effect was greatest at 0.1% and 1% serum. Cells were harvested and transplanted to recipient testes after 7 and 14 days of culture, and the number of colonies formed per 10⁵ cells placed in culture was determined (FIG. 11E). After 2 weeks exposure to serum, the stem cell activity was reduced to 20-50% of that in serum-free conditions. No serum concentration evaluated was able to enhance stem cell proliferation above that obtained in serum-free medium, indicating the balance of negative and positive influence of serum does not favor SSCs self-renewal even in a relatively pure population of stem cells.

Expression of gp 130, the shared signal transducing receptor component for the interleukin-6 family of cytokines, and a low revel of c-Kit receptor tyrosine kinase expression on the cell surface was detected (FIG. 11B), suggesting that leukemia inhibitory factor (LIF) and stem cell factor (SCF) may affect proliferation of SSCs in vitro. These growth factors have also been shown to have crucial roles in the self-renewal and differentiation of ES cells and PGCs (23,24). In addition to LIP and SCF, the effect of epidermal growth factor (EGF), insulin-like growth factor-I (IGF-1), and Noggin, an antagonist for bone morphogenetic proteins, were also investigated on proliferating SSCs. This is because signaling pathways activated by these soluble factors have been suggested to influence self-renewal of stem cells (Jamora et al., Nature, 422:317 (2003), Musaro et al., Proc. Natl. Acad. Sci. U.S.A., 101:1206 (2004), Reynolds et al., Dev. Biol., 175:1 (1996)). The factors were added individually in cultures of C57xROSA-derived SSCs maintained with GDNF, soluble GFRal and bFGF. SSCs were cultured with these factors for 6 weeks and transplanted into recipient testes to evaluate the stem cell activity (FIG. 13). Only, IGF-I significantly increased stem cell activity (2.8-fold, p<0.0005) suggesting that proliferation of SSCs can be modulated by IGF-1.

Finally, it was examined whether cultured SSCs could restore fertility when transplanted into infertile recipients. W mutant mice were used as recipients for transplantation; W mice are congenitally infertile because of a mutation in the c-Kit receptor. In addition, it has been shown previously that the W pup testis environment enhances efficient colonization by transplanted stem cells (Shinohara et al., Proc. Natl. Acad. Sci. U.S.A., 98:6186 (2001)). SSCs carrying a green fluorescent protein (GFP) reporter gene were cultured 11 weeks and then transplanted into pup testes of W mutant mice. Recipients were mated to C57BL/6J x SJL female mice, and 110 days after transplantation, progeny were produced (FIG. 11F). Similar results were obtained using SSCs carrying the lacZ reporter gene. There were no abnormalities in the progeny, and they were fertile. These results demonstrate that in vitro proliferating SSCs are able to produce functionally normal spermatozoa following transplantation.

This study has defined specific growth requirements for in vitro expansion of mouse SSCs. Stem cells from DBAxROSA mice required only GDNF indicating that GDNF is an essential growth factor for SSCs in vitro. The complementary role of soluble GFRal in mouse strains other than DBA/2J supports the critical role of this signaling pathway. Elucidation of the signaling cascades following activation of GDNF receptors in SSCs will now allow a critical assessment of the cellular and molecular mechanisms required for self-renewal of these stem cells. Identical or similar mechanisms are likely to function in most mammalian species, because SSCs of many species are known to proliferate on the basement membrane of mouse seminiferous tubules (Brinster, Science, 296:2174 (2002)). The serum-free in vitro system provides a powerful approach to understand signaling events involved in self-renewal of SSCs and to compare these mechanisms to other stem cells. While SSCs and ES cells share some characteristics such as Oct-4 expression, significant differences exist, e.g. AP activity and response to FBS. In addition, SSCs cultured for long periods do not generate tumors when transplanted to nude mice; whereas ES cells produce highly invasive teratocarcinomas when injected into mice (Evans et al., Nature, 292:154 (1981), Martin, Proc. Natl. Acad. Sci. U.S.A., 78:7634 (1981)). Therefore, fundamental differences must exist between these two related stem cells with respect to their self-renewal signaling pathway.

In adults, SSCs are the only stem cells that are able to transmit genetic information to subsequent generations. Therefore, SSCs provide an alternative method to modify the germline of animals, and in vitro proliferation of SSCs will make possible sophisticated genetic manipulation of these cells, including targeted modification.

Moreover, the large number of stem cells that can be generated in culture will be a powerful resource for gene analysis and functional genomics. Finally, by controlling the self-renewal vs. differentiation decision of SSCs, development of culture conditions that allow SSCs to produce functional gametes in vitro will create a valuable model for studying the molecular and cellular biology of male germ cell differentiation and will allow development of new therapeutic strategies for infertility.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of enriching spermatogonial stem cells (SSCs) from a population of testis-derived cells containing at least one SSC, said method comprising:
a) providing an antibody specific for a SSC cell-surface marker selected from the group consisting of Thy-1 and α6-integrin;
b) contacting said population of testis-derived cells with said antibody under conditions suitable for formation of an antibody-SSC complex; and
c) substantially separating said antibody-SSC complex from said population of testis-derived cells;
thereby enriching said SSCs.

2. (canceled)

3. A method of enriching spermatogonial stem cells (SSCs) from a population of testis-derived cells containing at least one SSC, said method comprising:
a) providing a first antibody specific for the SSC cell surface marker Thy-1;
b) providing a second antibody specific for an SSC cell surface marker other than Thy-1;
c) contacting said population of testis-derived cells with said first antibody under conditions suitable for formation of an antibody-SSC complex;
d) substantially separating said first antibody-SSC complex from said population of testis-derived cells, thereby creating a first antibody-SSC complex population of cells;
e) contacting said first antibody-SSC complex population of cells with said second antibody under conditions suitable for formation of a second antibody-SSC complex; and
f) substantially separating said second antibody-SSC complex from said population of testis-derived cells;
thereby enriching said SSCs.

4. The method of claim 1, wherein said SSC is a human SSC.

5. The method of claim 1, wherein said SSC is derived from an organism selected from the group consisting of a mouse, a rat, a monkey, a baboon, a cow, a pig and a dog.

6. The method of claim 1, wherein said antibody is selected from the group consisting of an isolated antibody, a biological sample comprising an antibody, an antibody bound to a physical support and a cell-bound antibody.

7. The method of claim 1, wherein said antibody is selected from the group consisting of a plyclonal antibody, a monoclonal antibody, a humanized antibody, a synthetic antibody, and combinations thereof, or biologically active fragments, functional equivalents, derivatives, and allelic or species variants thereof.

8. The method of claim 7, wherein said biologically active fragment is selected from the group consisting of a Fab fragment, a F(ab′)₂ fragment, and a Fv fragment.

9. The method of claim 6, wherein said physical support is selected from the group consisting of a microbead, a magnetic bead, a panning surface, a dense particle for density centrifugation, an adsorption column and an adsorption membrane.

10. The method of claim 1, wherein said antibody-SSC complex is substantially separated from said population of testis-derived cells by a method selected from the group consisting of fluorescence activated cell sorting (FACS) and magnetic activated cell sorting (MACS).

11. (canceled)

12. A method of detecting an SSC in a population of testis-derived cells, said method comprising:
a) providing an antibody specific for at least one cell surface marker selected from the group consisting of Thy-1, epithelial glycoprotein-2 (EpCAM), neural cell adhesion molecule (NCAM), glial cell-derived neurotrophic factor family receptor alpha-1 (GFRα1) and cell adhesion marker CD24 (CD24);
b) contacting said population of testis-derived cells with said antibody under conditions suitable for formation of an antibody-SSC complex; and
c) detecting said antibody-SSC complex;
thereby detecting said SSC in said population of testis-derived cells.

13. The method of claim 12, wherein said population of testis-derived cells is derived from a source selected from the group consisting of adult testis and neonate testis.

14. The method of claim 13, wherein said population of testis-derived cells is a population of human cells.

15. The method of claim 12, wherein said antibody is selected from the group consisting of an isolated antibody, a biological sample comprising an antibody, an antibody bound to a physical support and a cell-bound antibody.

16. The method of claim 12, wherein said antibody is selected from the group consisting of a polyclonal antibody, a monoclonal antibody, a humanized antibody, a synthetic antibody, and combinations thereof, or biologically active fragments, functional equivalents, derivatives, and allelic or species variants thereof.

17. The method of claim 16, wherein said biologically active fragment is selected from the group consisting of a Fab fragment, a F(ab′)₂ fragment, and a Fv fragment.

18. The method of claim 12, wherein said cells are derived from a source selected from the group consisting of mouse wild type adult testis, mouse pup testis, mouse neonate testis, and mouse cryptorchid adult testis.

19. The method of claim 12, wherein said cells are derived from a human.

20. A serum-free culture system for support of SSC maintenance, said culture system comprising:
a) enriched SSCs;
b) serum-free defined culture medium; and
c) mitotically-inactivated fibroblast feeder cells.

21. A serum-free culture system for support of SSC proliferation, said culture system comprising:
a) at least one SSC;
b) serum-free defined culture medium; and
c) mitotically-inactivated mouse fibroblast cell line STO (“STO”) feeder cells.

22. The culture system of claim 20, wherein said culture system further comprises at least one growth factor selected from the group consisting of SCF, GDNF, GFRα1, LIF, bFGF, EGF and IGF-I.

23. The culture system of claim 20, said medium comprising at least one medium selected from the group consisting of minimal essential medium-alpha (MEM ), Ham's F10 culture medium, RPMI bicarbonate-buffered medium, and Dulbecco's MEM Ham's Nutrient Mixture F-12 (DMEM/F12).

24. A composition comprising a population of enriched SSCs, wherein said enriched SSCs express a Thy-1 marker.

25. A composition comprising a population of Thy-1-enriched SSCs.

26. The composition of claim 25, wherein said population of Thy-1-enriched SSCs is substantially homogeneous for SSCs expressing a Thy-1 marker.

27. The composition of claim 24, wherein said population of enriched SSCs is substantially homogeneous for SSCs expressing a Thy-1 marker.

28. The method of claim 34, wherein said enriched SSCs are Thy-1-enriched SSCs.

29. The method of claim 34, wherein said population of enriched SSCs are administered to the lumen of a seminiferous tubule of said recipient mammal.

30. The method of claim 34, wherein said recipient mammal is infertile.

31. The method of claim 34, wherein said recipient mammal is selected from the group consisting of a rodent, a primate, a dog, a cow, a pig and a human.

32. The method of claim 31, wherein said rodent is selected from the group consisting of a mouse and a rat.

33. The method of claim 31, wherein said primate is a baboon.

34. A method of generating at least one progeny mammal, said method comprising:
a) administering a population of enriched SSCs to a testis of a male recipient mammal;
b) allowing said enriched SSCs to generate a colony of spermatogenic cells in said recipient mammal; and
c) mating said recipient mammal with a female mammal of the same species as said recipient mammal.

35. A method of determining the effect of a growth factor on an SSC, said method comprising:
a) providing a serum-free SSC culture system comprising a first population of enriched SSCs, serum-free defined culture medium, and a population of mitotically inactivated STO feeder cells;
b) contacting said culture system with at least one growth factor;
c) assessing the activity of said first population of enriched SSCs; and
d) comparing said activity of said first population of enriched SSCs with a second population of enriched SSCs, wherein said second population of said enriched SSCs is cultured in a growth factor-free culture system that is otherwise identical to the culture system comprising said first population of enriched SSCs;
wherein a higher level of SSC activity in said population of first enriched SSCs is an indication that said growth factor enhances the activity of an SSC and wherein a lower level of SSC activity in said population of first enriched SSCs is an indication that said growth factor inhibits the activity of an SSC, thereby determining the effect of a growth factor on an SSC.

36. (canceled)

37. The method of claim 35, wherein said growth factor is selected from the group consisting of bFGF, IGF1, GDNF and GFRα1 .

38. The method of claim 35, wherein said growth factor is selected from the group consisting of LIF, bFGF, EGF and IGF-I.

39. A method of maintaining at least one SSC in a serum-free culture system, said method comprising:
a) providing a culture system comprising serum-free defined culture medium and mitotically-inactivated STO feeder cells; and
b) adding at least one enriched SSC to said culture system.

40. The method of claim 39, wherein said method further comprises essentially eliminating inhibitory testis somatic cells and germ cells from said culture system.

41. A method of proliferating at least one SSC in a serum-free culture system, said method comprising:
a) providing a culture system comprising serum-free defined culture medium and mitotically-inactivated STO feeder cells; and
b) adding at least one enriched SSC to said culture system.

42. The method of claim 41, wherein said method further comprises essentially eliminating inhibitory testis somatic cells and germ cells from said culture system.

43. (canceled)

44. (canceled)

45. (canceled)

46. A method of proliferating at least one SSC in a serum-free culture system, said method comprising:
a) providing a culture system comprising serum-free defined culture medium and mitotically-inactivated STO feeder cells;
b) adding at least one SSC to said culture system; and
c) stimulating at least one GDNF cell-signaling pathway in said SSC;
wherein the stimulation of said GDNF cell-signaling pathway is effected by using at least one of the factors selected from the group consisting of GDNF, GFRaI and bFGF.

47. The method of claim 46, wherein said SSC is an enriched SSC.

48. A method of proliferating at least one SSC in a culture system, said method comprising:
a) providing a culture system comprising a culture medium and mitotically-inactivated STO feeder cells; and
b) adding at least one enriched SSC to said culture system.

49. The method of claim 48, wherein said method further comprises essentially eliminating inhibitory testis somatic cells and germ cells from said culture system.

50. (canceled)

51. A method of proliferating at least one SSC in a culture system, said method comprising:
a) providing a culture system comprising a culture medium and mitotically-inactivated STO feeder cells;
b) adding at least one enriched SSC to said culture system; and
c) stimulating at least one GDNF cell-signaling pathway in said enriched SSC.

52. The method of claim 52, wherein the stimulation of said GDNF cell-signaling pathway is effected by using at least one of the factors selected from the group consisting of GDNF, GFRα1 and bFGF.

53. A kit for maintaining at least one SSC in a serum-free culture system, said kit comprising:
a) a culture system comprising serum-free defined culture medium and mitotically-inactivated STO feeder cells;
b) an applicator, and
c) instructional material;
wherein said instructional material comprises instructions for the use of said kit to maintain at least one SSC in said serum-free culture system.

54. A kit for proliferating at least one SSC in a serum-free culture system, said kit comprising:
a) a culture system comprising serum-free defined culture medium and mitotically-inactivated STO feeder cells;
b) an applicator, and
c) instructional material;
wherein said instructional material comprises instructions for the use of said kit to proliferate at least one SSC in said serum-free culture system.

55. A kit for administering a population of enriched SSC to a mammal, said kit comprising:
a) a culture system comprising serum-free defined culture medium and mitotically-inactivated STO feeder cells;
b) an applicator, and
c) instructional material;
wherein said instructional material comprises:
a) instructions for the use of said kit to proliferate at least one SSC in said serum-free culture system; and
b) instructions for the applicator-based administration of said enriched SSC to said mammal.

56. (canceled)

57. (canceled)

58. A progeny animal produced according to the method of claim 34.

59. The progeny animal of claim 58, wherein said enriched SSCs contain at least one genetic mutation, wherein said genetic mutation is created using recombinant techniques.
